(19) United States
(12) Patent Application Publication
(10) Pub. No.: US 2011/0144311 A1
(43) Pub. Date: Jun. 16, 2011

(54) METHODS FOR PURIFYING ANTIBODIES USING PROTEIN A AFFINITY CHROMATOGRAPHY

(76) Inventors: Rebecca Chmielowski, Clark, NJ (US); Erin Green-Trexler, Sanatoga, PA (US); David Roush, Colts Neck, NJ (US)

(21) Appl. No.: 13/058,301

(22) PCT Filed: Aug. 10, 2009
(86) PCT No.: PCT/US09/53260

§ 371 Date: Feb. 9, 2011

Related U.S. Application Data
(60) Provisional application No. 61/188,903, filed on Aug. 14, 2008.

(51) Int. Cl. C07K 1/22

(52) U.S. Cl. 530/388.1

(57) ABSTRACT

This invention provides a method for purifying a monomeric monoclonal antibody which comprises contacting the sample, wherein the sample comprises the monomeric monoclonal antibody, host cell impurities, dimers, and higher order aggregates, with a Protein A affinity chromatography column; eluting the monomeric monoclonal antibody from the Protein A affinity chromatography column with an elution buffer; and collecting one or more fractions of the monomeric monoclonal antibody to form a Protein A product pool, wherein the product pool comprises less than 5% higher order aggregate, and has a pH from about 3.2 to about 4.5, thereby purifying the monomeric monoclonal antibody from the sample. This invention also provides a method for purifying a monomeric monoclonal antibody which comprises eluting with acetate or citrate, optionally in the presence of amino acids. This invention also provides a method for purifying a monomeric monoclonal antibody which comprises conducting the method within certain temperature ranges.

Throughout this application, various references are referred to by Arabic numerals in parentheses. Disclosures of these publications in their entireties are hereby incorporated by reference into the application to more fully describe the state of the art to which this invention pertains. Full bibliographic citation for these references may be found immediately preceding the claims.

### BACKGROUND OF THE INVENTION

Over the past decade, the applications for therapeutic monoclonal antibodies (mAbs) have significantly increased. MAb stability represents a current challenge in the purification and formulation of these proteins. MAb instability leads to high levels of aggregated mAb in protein formulations, which can have several disadvantages including changing protein activity and potentially leading to undesirable immunological responses in patients.

Protein A affinity chromatography is a powerful and widely-used tool for purifying antibodies. In order to elute a protein or antibody from the Protein A resin, acidic conditions are required due to the high affinity of the monoclonal antibodies to the resin. Exposure to these acidic conditions can result in the formation of protein aggregates. Some strategies to address aggregation during Protein A chromatography have been previously described in the literature (1, 2, 3, 4, 5, 6). Furthermore, a low pH hold step following elution is required for viral inactivation and can also result in the formation of protein aggregates (7, 8, 9).

Previously, one approach to reducing protein aggregation in mAb formulations was to use additional chromatography steps. This solution is both expensive in terms of materials and processing time; and it also results in product losses with each step, which can reduce the overall yield of mAb product.

Another previous approach to reducing protein aggregation in mAb formulations was to use advanced chromatography methods, such as peak cutting, to reduce the amount of protein aggregation following the affinity chromatography step. These approaches are time-consuming, and they are often unsuccessful necessitating additional chromatography steps to produce mAb formulations suitable for human use.

Yet another previous approach to reducing protein aggregation in mAb formulations was to use stabilizing agents, which can have several disadvantages including, changes in protein activity, difficulty in further purification steps, and potentially undesirable immunological responses in patients.

The methods that are the subject of the present invention address the need for simpler and less expensive processes for reducing protein aggregation in monoclonal antibody formulations in order to purify monomeric monoclonal antibodies suitable for human use.

### SUMMARY OF THE INVENTION

This invention provides a first method for purifying a monomeric monoclonal antibody from a sample, wherein the sample comprises the monomeric monoclonal antibody, host cell impurities, dimers, and higher order aggregates, comprising: (a) contacting the sample with a Protein A affinity chromatography column; (b) eluting the monomeric monoclonal antibody from the Protein A affinity chromatography column with an elution buffer; and (c) collecting one or more fractions of the monomeric monoclonal antibody from step (b) to form a Protein A product pool, wherein the product pool (i) comprises less than 5% higher order aggregate, and (ii) has a pH from about 3.5 to about 4.5, thereby purifying the monomeric monoclonal antibody from the sample.

This invention provides a second method for purifying a monomeric monoclonal antibody from a sample, wherein the sample comprises the monomeric monoclonal antibody, host cell impurities, dimers, and higher order aggregates, comprising: (a) contacting the sample with a Protein A affinity chromatographic column at a temperature from about 15° C. to about 27° C.; (b) eluting the monomeric monoclonal antibody from the Protein A affinity chromatographic column with an elution buffer comprising citrate at a concentration from about 0.030 M to about 0.085 M; and (c) collecting one or more fractions of the monomeric monoclonal antibody from step (b) to form a Protein A product pool, wherein the product pool (i) comprises less than 5% higher order aggregate, and (ii) has a pH from about 3.5 to about 4.0, thereby purifying the monomeric monoclonal antibody from the sample.

This invention provides a third method for purifying a monomeric monoclonal antibody from a sample, wherein the sample comprises the monomeric monoclonal antibody, host cell impurities, dimers, and higher order aggregates, comprising: (a) contacting the sample with a Protein A affinity chromatographic column at a temperature from about 15° C. to about 27° C.; (b) eluting the monomeric monoclonal antibody from the Protein A affinity chromatographic column with an elution buffer comprising acetate at a concentration from about 0.050 M to about 0.200 M; and (c) collecting one or more fractions of the monomeric monoclonal antibody from step (b) to form a Protein A product pool, wherein the product pool (i) comprises less than 5% higher order aggregate, and (ii) has a pH from about 3.5 to about 4.5, thereby purifying the monomeric monoclonal antibody from the sample.

### BRIEF DESCRIPTION OF THE DRAWINGS

FIG. 1 shows the rate of higher order aggregate formation at a PAP pool pH of 6.1 as a function of time at 4° C., 17° C. and 37° C. for an anti-DKK-1 monoclonal antibody (SEQ ID NO:1 and SEQ ID NO:2 shown in FIG. 22). In the figure, □=pH 6.1 at 4° C., ∘=pH 6.1 at 17° C., and Δ=pH 6.1 at 37° C.

FIG. 2 shows the rate of higher order aggregate formation at a PAP pool pH of 3.5 as a function of time at 4° C., 17° C. and 37° C. for the anti-DKK-1 monoclonal antibody (SEQ ID NO:1 and SEQ ID NO:2 shown in FIG. 22). In the figure, □=pH 3.5 at 4° C., ∘=pH 3.5 at 17° C., and Δ=pH 3.5 at 37° C.

FIG. 3 shows the rate of dimer formation at a PAP pool pH of 6.1 as a function of time at 4° C., 17° C. and 37° C. for the anti-DKK-1 monoclonal antibody. In the figure, □=pH 6.1 at 4° C., ∘=pH 6.1 at 17° C., and Δ=pH 6.1 at 37° C.

FIG. 4 shows the rate of dimer formation for the anti-DKK-1 monoclonal antibody at a PAP pool pH of 3.5 as a function of time at 4° C., 17° C. and 37° C. In the figure, □=pH 3.5 at 4° C., and ∘=pH 3.5 at 17° C.

FIG. 5 shows the rate of higher order aggregate formation for the anti-DKK-1 monoclonal antibody at a PAP pool pH of 3.5 as a function of time at 25° C. and 30° C. In the figure, □=pH 3.5 at 25° C., and ∘=pH 3.5 at 30° C.

FIG. 6 shows the rate of dimer formation for the anti-DKK-1 monoclonal antibody at a PAP pool pH of 3.5 as a function of time at 25° C. and 30° C. In the figure, □=pH 3.5 at 25° C., and ∘=pH 3.5 at 30° C.

FIG. 7 shows the rate of higher order aggregate formation at a pH of 4.0, 4.5, and 5.0 as a function of time at 21° C. for the anti-DKK-1 monoclonal antibody. In the figure, □=pH 4.0 at 21° C., ∘=pH 4.5 at 21° C., and Δ=pH 5.0 at 21° C.

FIG. 8 shows the rate of higher order aggregate formation at a pH of 4.0, 4.5, and 5.0 as a function of time at 30° C. for the anti-DKK-1 monoclonal antibody. In the figure, □=pH 4.0 at 30° C., ∘=pH 4.5 at 30° C., and Δ=pH 5.0 at 30° C.

FIG. 9 shows the rate of dimer formation at a pH of 4.0, 4.5, and 5.0 as a function of time at 21° C. for the anti-DKK-1 monoclonal antibody. In the figure, □=pH 4.0 at 21° C., ∘=pH 4.5 at 21° C., and Δ=pH 5.0 at 21° C.

FIG. 10 shows the rate of dimer formation at a pH of 4.0, 4.5, and 5.0 as a function of time at 30° C. for the anti-DKK-1 monoclonal antibody. In the figure, □=pH 4.0 at 30° C., ∘=pH 4.5 at 30° C., and Δ=pH 5.0 at 30° C.

FIG. 11 shows levels of higher order aggregates versus time at 4° C., 17° C., 25° C. and 30° C. for the anti-DKK-1 monoclonal antibody PAP at pH 3.5. In the figure, ▪=30° C., ♦=25 ° C., ▴=17° C., and x=4° C.

FIG. 12 shows higher order aggregate formation as a function of time and temperature at pH 3.91 and 50 mM citrate concentration for the anti-DKK-1 monoclonal antibody. In the figure, ⋄=pH 3.91 at 4° C., □=pH 3.91 at 15° C., Δ=pH 3.91 at 20° C., and x=pH 3.91 at 24° C.

FIG. 13 shows higher order aggregate formation as a function of time at 50 mM and 100 mM citrate concentration at room temperature for the anti-DKK-1 monoclonal antibody. In the figure, ▪=pH 3.5 at 25° C., and ♦=pH 3.91 at 24° C.

FIG. 14 shows higher order aggregate formation as a function of citrate concentration and time at 25° C. for the anti-DKK-1 monoclonal antibody. In the figure, [symbol] =60 mM citrate at pH 3.8, [symbol] =75 mM citrate at pH 3.6, [symbol] =100 mM citrate at pH 3.6, [symbol] =85 mM citrate at pH 3.4, and [symbol] =40 mM citrate at pH 3.4.

FIG. 15A shows the DSC profiles for the anti-DKK1 antibody in 30 mM, 60 mM and 100 mM citrate at pH 3.0.

FIG. 15B shows the DSC profiles for the anti-DKK1 antibody in 30 mM, 60 mM and 100 mM citrate at pH 3.5.

FIG. 15C shows the DSC profiles for the anti-DKK1 antibody in 30 mM, 60 mM and 100 mM citrate at pH 4.0.

FIG. 16A shows the DSC profiles for the anti-DKK1 antibody in 30 mM, 60 mM and 100 mM citrate at pH 4.5.

FIG. 16B shows the DSC profiles for the anti-DKK1 antibody in 30 mM, 60 mM and 100 mM citrate at pH 5.0.

FIG. 16C shows the DSC profiles for the anti-DKK1 antibody in 30 mM, 60 mM and 100 mM citrate at pH 5.5.

FIG. 16D shows the DSC profiles for the anti-DKK1 antibody in 30 mM, 60 mM and 100 mM citrate at pH 6.0.

FIG. 17 shows the rate of higher order aggregate for the anti-DKK-1 monoclonal antibody formation for 60 mM citrate elution with and without 50 mM arginine at 25° C. In the figure, ⋄=60 mM citrate+50 mM arginine at pH 3.5 and 25° C., and □=60 mM citrate only at pH 3.5 and 25° C.

FIG. 18 shows the rate of higher order aggregate formation for the anti-DKK-1 monoclonal antibody for 100 mM citrate elution with and without 250 mM arginine at 25° C. In the figure, ⋄=100 mM citrate+250 mM arginine at pH 3.5 and 25° C., and □=100 mM citrate only at pH 3.5 and 25° C.

FIG. 19 shows the rate of higher order aggregate formation for the anti-DKK-1 monoclonal antibody for various citrate concentrations as compared to phosphate buffer as a function of time at 25° C. In the figure, [symbol] =60 mM citrate, [symbol] =75 mM citrate, [symbol] =40 mM citrate, [symbol] =H₃PO₄, [symbol] =100 mM citrate, and [symbol] =85 mM citrate.

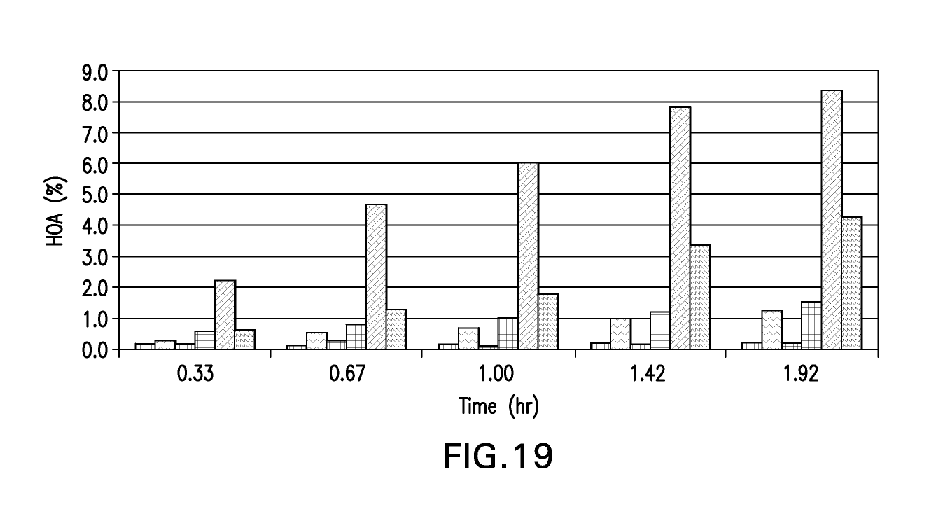

FIG. 20 shows the effect of monoclonal antibody concentration for the anti-DKK-1 monoclonal antibody on the rate of higher order aggregation at 25° C. over time in 15 mM citrate. In the figure, ♦=8 mg/mL anti-DKK-1 mAb, ▪=14 mg/mL anti-DKK-1 mAb, and ▴=34 mg/mL anti-DKK-1 mAb.

FIG. 21 shows the effect of monoclonal antibody concentration for the anti-DKK-1 monoclonal antibody on the rate of higher order aggregation over time at pH 4.0 in 15 mM citrate at 21° C. and 85 mM acetate at 25° C. In the figure, =5 mg/mL anti-DKK-1 mAb in acetate,+=11 mg/mL anti-DKK-1 mAb in acetate, Δ=37 mg/mL anti-DKK-1 mAb in acetate, and ♦=7 mg/mL anti-DKK-1 mAb in citrate.

FIG. 22 shows the anti-DKK-1 monoclonal antibody amino acid sequences for the heavy and light chains. (SEQ ID NO:1 and SEQ ID NO:2)

FIG. 23 shows the anti-ADDL #1 monoclonal antibody amino acid sequences for the heavy and light chains. (SEQ ID NO:3 and SEQ ID NO:4)

FIG. 24 shows the anti-ADDL #2 monoclonal antibody amino acid sequences for the heavy and light chains. (SEQ ID NO:5 and SEQ ID NO:6)

FIG. 25 shows the anti-hIL-13rα-1 monoclonal antibody amino acid sequences for the heavy and light chains. (SEQ ID NO:7 and SEQ ID NO:8)

FIG. 26 shows the alignment of the amino acid sequence from the IgG2m4 Fc region of the monoclonal antibody compared to that of the Fc regions from IgG1, IgG2, and IgG4.

### DETAILED DESCRIPTION OF THE INVENTION

### Definitions

The term “protein” or “polypeptide” as used herein shall mean a polypeptide made up of amino acid residues covalently linked together by peptide bonds.

As used herein, the terms “antibody,” “immunoglobulin,” and “immunoglobulin molecule” shall be used interchangeably. Each antibody has a unique structure that allows it to bind its specific antigen, but all antibodies have the same overall structure as described herein. The basic antibody structural unit is known to comprise a tetramer of subunits. Each tetramer has two identical pairs of polypeptide chains, each pair having one “light” chain (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function (10).

The term “Fc” fragment shall refer to the ‘fragment crystallized’ C-terminal region of the antibody containing the C_{H}2 and C_{H}3 domains. The term “Fab” fragment shall refer to the ‘fragment antigen binding’ region of the antibody containing the V_{H}, C_{H}1, V_{L} and C_{L} domains.

The term “monoclonal antibody” (mAb) as used herein shall refer to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each mAb is directed against a single determinant on the antigen. In addition to their specificity, monoclonal antibodies are advantageous in that they can be synthesized by hybridoma culture, uncontaminated by other immunoglobulins. The term “monoclonal” indicates the character of the antibody as being obtained from a substantially homogeneous population of antibodies, and is not to be construed as requiring production of the antibody by any particular method. For example, the monoclonal antibodies herein can be made by the hybridoma method first described by Kohler et al., (1975) Nature, 256:495, or may be made by recombinant DNA methods (11).

The term “monomeric monoclonal antibody” as used herein shall refer to an antibody molecule containing two heavy chains and two light chains, i.e. a monomer.

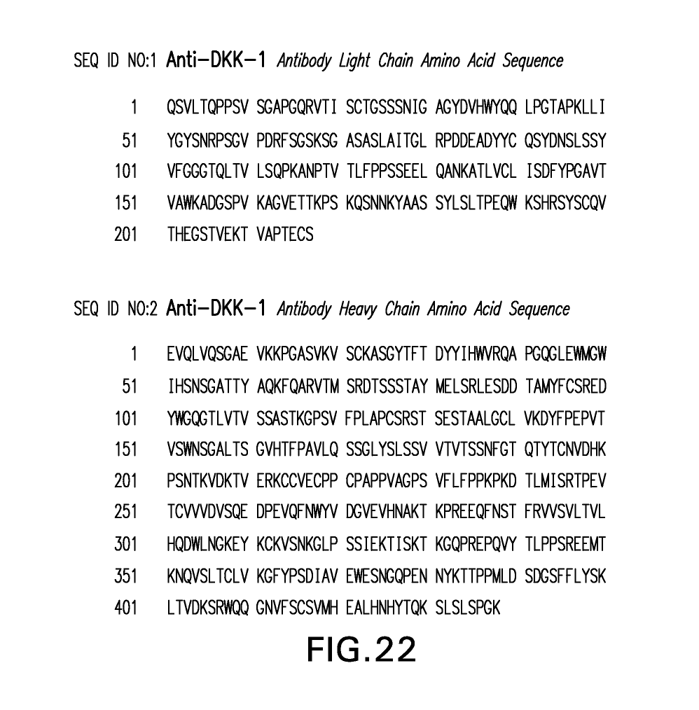

As used herein, an “anti-DKK-1” antibody shall mean a monoclonal antibody with the amino acid sequences for light and heavy chains as set forth in SEQ ID NO:1 and SEQ ID NO:2, see FIG. 22.

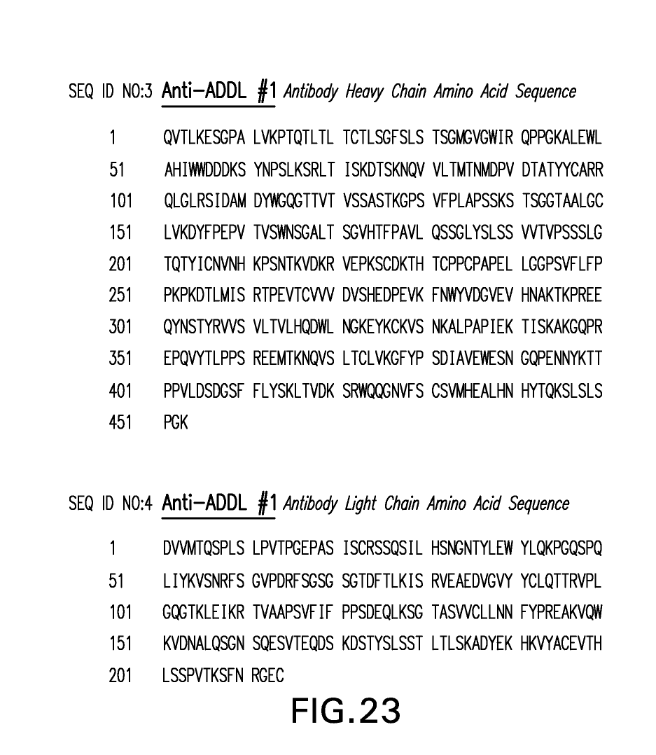

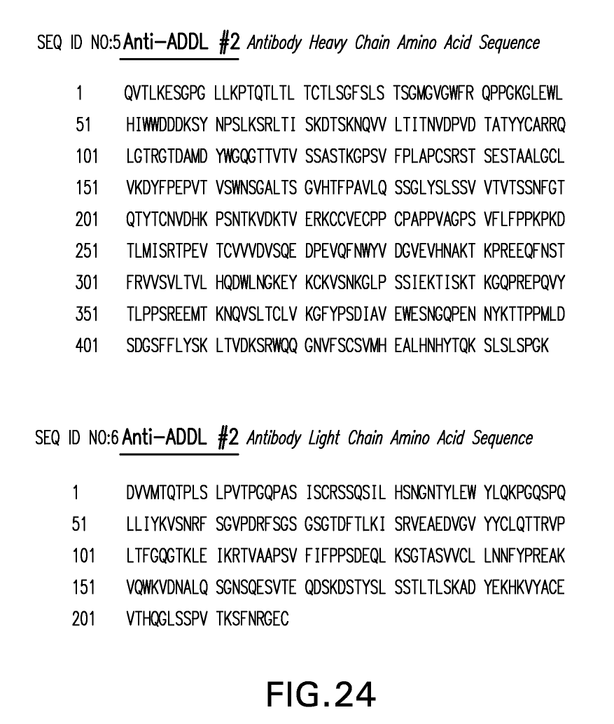

As used herein, an “anti-ADDL” antibody shall mean a monoclonal antibody with the amino acid sequences for heavy and light chains set forth here in SEQ ID NO:3 and SEQ ID NO:4 or SEQ ID NO:5 and SEQ ID NO:6, see FIG. 23 and FIG. 24, respectively.

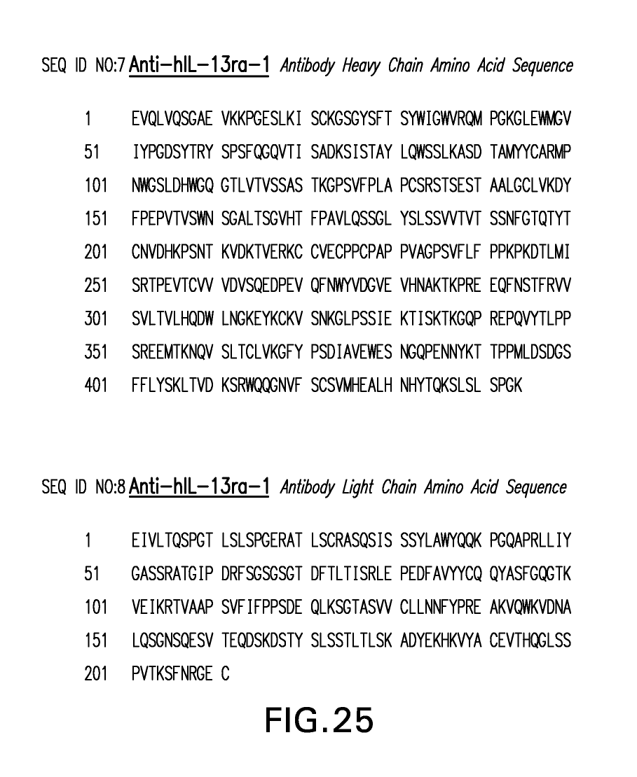

As used herein, an “anti-hIL-13rα1” antibody shall mean a monoclonal antibody with the amino acid sequences for heavy and light chains as set forth in SEQ ID NO:7 and SEQ ID NO:8, see FIG. 25.

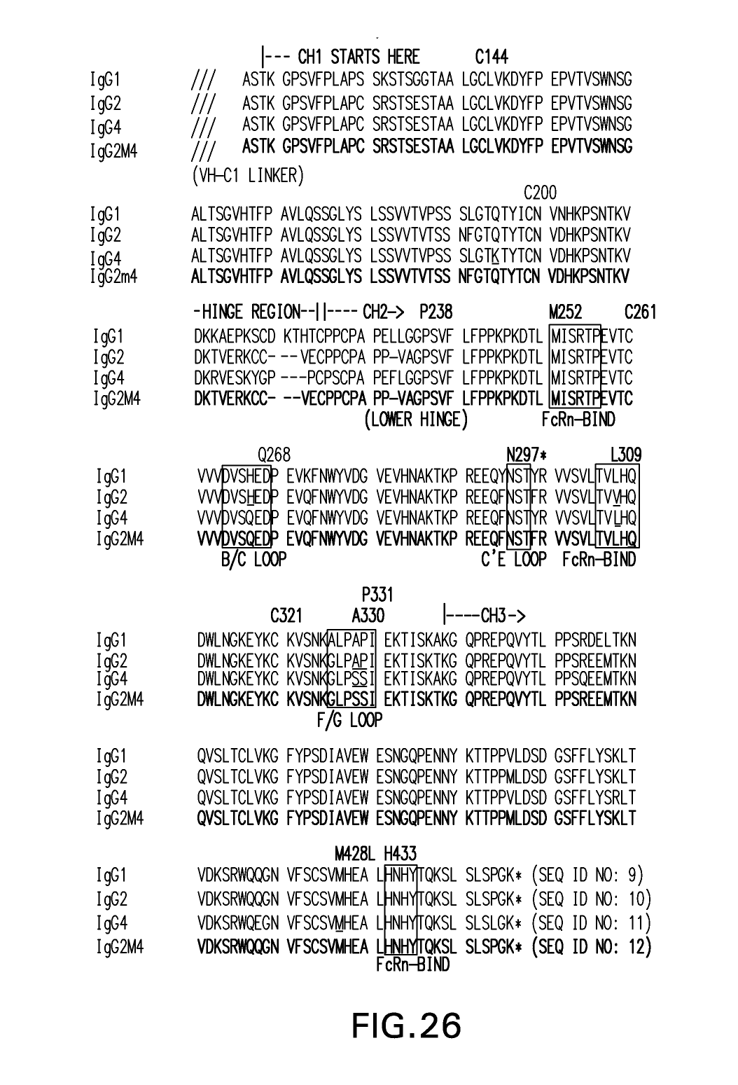

As used herein, a “modified IgG2 antibody” shall mean an antibody having an “IgG2m4” Fc region of a monoclonal antibody as represented by the amino acid sequence shown in FIG. 26.

The term “dimer” as used herein shall mean a biologic molecule consisting of two subunits called monomers. “Dimers” of the present invention shall mean a molecule containing two monomeric monoclonal antibodies.

The term “higher order aggregate” or “HOA” as used herein shall mean large oligomers of monomeric monoclonal antibodies, being typically greater than 300 kDa, i.e. dimer molecular weight.

The term “aggregate” or “aggregation” as used herein shall mean agglomeration or oligomerization of two or more individual molecules, i.e. protein aggregate or protein aggregation. Protein aggregates can be soluble or insoluble.

An “impurity” as used herein shall mean a material that is different from the desired protein product, such as protein aggregates. The impurity may be a variant of the desired protein or another protein. Specific examples of impurities herein include proteins from the host cell producing the desired protein, such as host proteins, leached protein A, DNA, RNA, etc. (See, e.g. U.S. Patent Application Publication No. US 2005/0038231).

The term “protein degradation” as used herein shall mean a monomeric monoclonal antibody or protein that degrades under a certain condition to form a dimer or higher order aggregate.

Primary recovery produces the feed stream to Protein A chromatography. One term used to describe this feed stream is called “depth filtered centrate” and as used herein shall mean a monoclonal antibody or protein solution that has been processed through centrifugation (cell and debris removal) and depth filtration (removal of fine debris that is <10 μm). In this invention, depth filtered product is used as the feed solution for the protein A affinity chromatography laboratory experiments and for production of various clinical lots.

The term “Protein A affinity chromatography” shall refer to the separation or purification of substances and/or particles using protein A, where the protein A is generally immobilized on a solid phase. Protein A is a 40-60 kD cell wall protein originally found in Staphylococcus aureas. The binding of antibodies to protein A resin is highly specific. Protein A binds with high affinity to the Fc region of immunoglobulins. It binds with high affinity to human IgG1 and IgG2 as well as mouse IgG2a and IgG2b. Protein A binds with moderate affinity to human IgM, IgA and IgE as well as to mouse IgG3 and IgG1. A protein comprising a C_{H}2/C_{H}3 region may be reversibly bound to, or adsorbed by, the protein A. Protein A affinity chromatography columns for use in protein A affinity chromatography herein include, but are not limited to, protein A immobilized on an agarose solid phase, for instance the MABSELECT™ or MABSURE™ columns (Amersham Biosciences Inc.); protein A immobilized on a polystyrene solid phase, for instance POROS 50A™ columns (Applied Biosystems Inc.).

“Sample”, when used in connection with the instant methods, includes, but is not limited to, any body tissue, blood, serum, plasma, cerebrospinal fluid, lymphocyte, exudate, or supernatant from a cell culture.

The terms “load” or “loading” shall mean the amount of a protein per unit volume.

The term “contacting” as used herein shall mean contacting a monoclonal antibody to Protein A resin in the Protein A affinity chromatography column.

The term “elution buffer” as used herein shall mean a buffer comprising a primary species, such as sodium citrate or sodium acetate, which is used to elute the antibody from the protein A affinity column.

As used herein, the term “citrate” shall mean the anionic species present in the elution buffer as derived from the corresponding acid or salt.

As used herein, the term “acetate” shall mean the anionic species present in the elution buffer as derived from the corresponding acid or salt.

The term “fraction” as used herein as in “collecting one or more fractions” shall mean the result of a separation process in which a certain quantity of a mixture (solid, liquid, solute or suspension) is divided up in a number of smaller quantities (“fractions”) in which the composition changes according to a gradient. Here, fractions are collected as the monomeric monoclonal antibody is eluted from the protein A affinity column.

The term “regeneration buffer” as used herein shall mean the buffer used to clean the column to remove bound impurities. For example, a high salt buffer, a NaOH-containing, or a phosphoric acid-containing buffer (13).

The term “column volume” or “CV” as used herein shall mean the volume of packed resin inside the column including any void volume. For example, if a 10 mL column is packed with 2 mL of resin, then one CV is 2 mL.

The term “residence time” as used herein shall mean an amount of time a portion of the product interacts with the resin.

The term “flow rate” as used herein shall mean the column volume divided by the residence time. For example, the flow rate for a column with 10 mL of resin at a specified residence time of 5 min would be as follows:

$$\frac{10 mL}{5 min} = 2 mL \text{/} min$$

The term “protein A product” or “PAP” as used herein shall mean the product which is eluted from the protein A affinity chromatography column using an acid such as sodium citrate or sodium acetate.

The term “quenched protein A product” or “QPAP” as used herein shall mean the addition of a base to the PAP, such as tris base or a phosphate solution, to raise the pH of the PAP from about pH 3.0 to 4.0 to about 6.0 to 7.5.

The term “yield” as used herein shall mean the amount of product recovered divided by the amount of product loaded onto the column multiplied by 100. For example, a column loaded with a solution that contained 100 grams of product, but from which 80 grams of product was recovered from the elution stream, would have an 80% yield.

The term “differential scanning calorimetry” or “DSC” as used herein shall mean a thermoanalytic technique that measures the difference in the amount of heat required to increase the temperature of a sample and reference as a function of temperature. For proteins, DSC provides information on the thermal stability of a protein and its individual domains and on the solubility of the unfolded forms of the protein.

The term “high pressure or performance liquid chromatography” or “HPLC” as used herein shall mean a form of column chromatography that utilizes high pressure to separate, identify, and quantify compounds. HPLC uses a column containing a stationary phase at a specified temperature, a pump for the mobile phase solution, and a detector to quantify each compound injected onto the column.

The term “high pressure size exclusion chromatography” or “HPSEC” shall mean a chromatographic method that uses high pressure (20 to 150 bar) to separate particles based on their molecular weight or hydrodynamic volume. In this invention, this technique is applied for the separation and quantification of monoclonal antibodies, dimers, and higher order aggregates.

The term “small scale” as used herein shall mean a protein A affinity column size of less than 300 mLs of resin.

The term “stabilizing agent” as used herein shall mean an agent, such as arginine proline, or histidine, which reduces the rate of protein aggregate formation.

The term “time zero sample” as used herein shall mean the starting time of the experiment, which represents immediately after the product has eluted from the resin.

### Embodiments of the Invention

This invention provides a first method for purifying a monomeric monoclonal antibody from a sample, wherein the sample comprises the monomeric monoclonal antibody, host cell impurities, dimers, and higher order aggregates, comprising: (a) contacting the sample with a Protein A affinity chromatography column; (b) eluting the monomeric monoclonal antibody from the Protein A affinity chromatography column with an elution buffer; and (c) collecting one or more fractions of the monomeric monoclonal antibody from step (b) to form a Protein A product pool, wherein the product pool (i) comprises less than 5% higher order aggregate, and (ii) has a pH from about 3.5 to about 4.5, thereby purifying the monomeric monoclonal antibody from the sample.

In one embodiment of the above method, the elution buffer is acetate or citrate.

In a further embodiment, the concentration of citrate in the elution buffer is from about 0.030 M to about 0.085 M. As used herein, “about” shall mean ±0.015 M.

In another further embodiment, the concentration of acetate is from about 0.050 M to about 0.200 M. As used herein, “about” shall mean ±0.015 M.

In another embodiment of the above method, the method is conducted at a temperature from about 4° C. to about 30° C. As used herein, “about” shall mean ±4° C.

In a further embodiment, the method is conducted at a temperature from about 15° C. to about 27° C. As used herein, “about” shall mean ±4° C.

In one embodiment, the monomeric monoclonal antibody is an IgG antibody.

In a further embodiment, the monomeric monoclonal antibody is an IgG1 or a modified IgG2 antibody.

In another embodiment the IgG1 antibody is an anti-ADDL antibody. One example is the anti-ADDL antibody of which the heavy and light chains are represented as SEQ ID NO:3 and SEQ ID NO:4 in FIG. 23 (See, e.g. PCT Intl. Appln. No. PCT/US2005/038125).

In yet a further embodiment, the modified IgG2 antibody is an IgG2m4 antibody (FIG. 26) (See, e.g., U.S. Ser. No. 11/581,931).

In another embodiment, the modified IgG2m4 antibody is an anti-DKK-1 antibody. One example is the anti-DKK-1 antibody of which the heavy and light chains are represented as SEQ ID NO:1 and SEQ ID NO:2 in FIG. 22 (See, e.g., U.S. Ser. No. 12/012,885).

In another embodiment, the modified IgG2m4 antibody is an anti-ADDL antibody. One example is the anti-ADDL antibody of which the heavy and light chains are represented as SEQ ID NO:5 and SEQ ID NO:6 in FIG. 24 (See, e.g., PCT Intl. Appln. No. PCT/US2006/040508).

In another embodiment, the modified IgG2m4 antibody is an anti-hIL-13rα-1 antibody. One example is the anti-hIL-13rα-1 antibody of which the heavy and light chains are represented as SEQ ID NO:7 and SEQ ID NO:8 in FIG. 25 (See, e.g., U.S. Ser. No. 11/875,017).

In one embodiment, an amino acid is added to the elution buffer at a concentration from about 50 mM to about 500 mM. As used herein, “about” shall mean ±0.015 M.

In a further embodiment, the amino acid used is histidine, proline, or arginine.

This invention provides a second method for purifying a monomeric monoclonal antibody from a sample, wherein the sample comprises the monomeric monoclonal antibody, host cell impurities, dimers, and higher order aggregates, comprising: (a) contacting the sample with a Protein A affinity chromatographic column at a temperature from about 15° C. to about 27° C.; (b) eluting the monomeric monoclonal antibody from the Protein A affinity chromatographic column with an elution buffer comprising citrate at a concentration from about 0.030 M to about 0.085 M; and (c) collecting one or more fractions of the monomeric monoclonal antibody from step (b) to form a Protein A product pool, wherein the product pool (i) comprises less than 5% higher order aggregate, and (ii) has a pH from about 3.5 to about 4.0, thereby purifying the monomeric monoclonal antibody from the sample.

In one embodiment of the above method, the elution buffer is acetate or citrate.

In a further embodiment, the concentration of citrate in the elution buffer is from about 0.030 M to about 0.085 M. As used herein, “about” shall mean ±0.015 M.

In another further embodiment, the concentration of acetate is from about 0.050 M to about 0.200 M. As used herein, “about” shall mean ±0.015 M.

In another embodiment of the above method, the method is conducted at a temperature from about 4° C. to about 30° C. As used herein, “about” shall mean ±4° C.

In a further embodiment, the method is conducted at a temperature from about 15° C. to about 27 ° C. As used herein, “about” shall mean ±4° C.

In one embodiment, the monomeric monoclonal antibody is an IgG antibody.

In a further embodiment, the monomeric monoclonal antibody is an IgG1 or a modified IgG2 antibody.

In another embodiment the IgG1 antibody is an anti-ADDL antibody. One example is the anti-ADDL antibody of which the heavy and light chains are represented as SEQ ID NO:3 and SEQ ID NO:4 in FIG. 23 (See, e.g. PCT Intl. Appln. No. PCT/US2005/038125).

In yet a further embodiment, the modified IgG2 antibody is an IgG2m4 antibody (FIG. 26) (See, e.g., U.S. Ser. No. 11/581,931).

In another embodiment, the modified IgG2m4 antibody is an anti-DKK-1 antibody. One example is the anti-DKK-1 antibody of which the heavy and light chains are represented as SEQ ID NO:1 and SEQ ID NO:2 in FIG. 22 (See, e.g., U.S. Ser. No. 12/012,885).

In another embodiment, the modified IgG2m4 antibody is an anti-ADDL antibody. One example is the anti-ADDL antibody of which the heavy and light chains are represented as SEQ ID NO:5 and SEQ ID NO:6 in FIG. 24 (See, e.g., PCT Intl. Appln. No. PCT/US2006/040508).

In another embodiment, the modified IgG2m4 antibody is an anti-hIL-13rα-1 antibody. One example is the anti-hIL-13rα-1 antibody of which the heavy and light chains are represented as SEQ ID NO:7 and SEQ ID NO:8 in FIG. 25 (See, e.g., U.S. Ser. No. 11/875,017).

In one embodiment, an amino acid is added to the elution buffer at a concentration from about 50 mM to about 500 mM. As used herein, “about” shall mean ±0.015 M.

In a further embodiment, the amino acid used is histidine, proline, or arginine.

This invention provides a third method for purifying a monomeric monoclonal antibody from a sample, wherein the sample comprises the monomeric monoclonal antibody, host cell impurities, dimers, and higher order aggregates, comprising: (a) contacting the sample with a Protein A affinity chromatographic column at a temperature from about 15° C. to about 27° C.; (b) eluting the monomeric monoclonal antibody from the Protein A affinity chromatographic column with an elution buffer comprising acetate at a concentration from about 0.050 M to about 0.200 M; and (c) collecting one or more fractions of the monomeric monoclonal antibody from step (b) to form a Protein A product pool, wherein the product pool (i) comprises less than 5% higher order aggregate, and (ii) has a pH from about 3.5 to about 4.5, thereby purifying the monomeric monoclonal antibody from the sample.

In one embodiment of the above method, the elution buffer is acetate or citrate.

In a further embodiment, the concentration of citrate in the elution buffer is from about 0.030 M to about 0.085 M. As used herein, “about” shall mean ±0.015 M.

In another further embodiment, the concentration of acetate is from about 0.050 M to about 0.200 M. As used herein, “about” shall mean ±0.015 M.

In another embodiment of the above method, the method is conducted at a temperature from about 4° C. to about 30° C. As used herein, “about” shall mean ±4° C.

In a further embodiment, the method is conducted at a temperature from about 15° C. to about 27 ° C. As used herein, “about” shall mean ±4° C.

In one embodiment, the monomeric monoclonal antibody is an IgG antibody.

In a further embodiment, the monomeric monoclonal antibody is an IgG1 or a modified IgG2 antibody.

In another embodiment the IgG1 antibody is an anti-ADDL antibody. One example is the anti-ADDL antibody of which the heavy and light chains are represented as SEQ ID NO:3 and SEQ ID NO:4 in FIG. 23 (See, e.g. PCT Intl. Appln. No. PCT/US2005/038125).

In yet a further embodiment, the modified IgG2 antibody is an IgG2m4 antibody (FIG. 26) (See, e.g., U.S. Ser. No. 11/581,931).

In another embodiment, the modified IgG2m4 antibody is an anti-DKK-1 antibody. One example is the anti-DKK-1 antibody of which the heavy and light chains are represented as SEQ ID NO:1 and SEQ ID NO:2 in FIG. 22 (See, e.g., U.S. Ser. No. 12/012,885).

In another embodiment, the modified IgG2m4 antibody is an anti-ADDL antibody. One example is the anti-ADDL antibody of which the heavy and light chains are represented as SEQ ID NO:5 and SEQ ID NO:6 in FIG. 24 (See, e.g., PCT Intl. Appln. No. PCT/US2006/040508).

In another embodiment, the modified IgG2m4 antibody is an anti-hIL-13rα-1 antibody. One example is the anti-hIL-13rα-1 antibody of which the heavy and light chains are represented as SEQ ID NO:7 and SEQ ID NO:8 in FIG. 25 (See, e.g., U.S. Ser. No. 11/875,017).

In one embodiment, an amino acid is added to the elution buffer at a concentration from about 50 mM to about 500 mM. As used herein, “about” shall mean ±0.015 M.

In a further embodiment, the amino acid used is histidine, proline, or arginine.

In a further embodiment of each of the above-described methods, the Protein A product pool has a pH of 3.2 or greater.

This invention will be better understood from the Examples which follow. However, one skilled in the art will readily appreciate that the specific methods and results discussed are merely illustrative of the invention as described more fully in the claims which follow thereafter.

### EXAMPLE 1

### Temperature Reduction for Reducing Levels of Protein Aggregates During Protein A Affinity Chromatography Elution and Subsequent Low pH Hold

To characterize the temperature and pH dependence of protein aggregation, the effect of temperature and pH on the PAP elution pool containing citrate (100 mM, pH 3.5) was evaluated with respect to an anti-DKK-1 monoclonal antibody. The same procedure can be utilized with other mAbs such as anti-ADDL monoclonal antibodies.

### Materials and Methods

Small-Scale: All small-scale experiments were performed using an AKTA EXPLORER 100™. Phosphate, citrate, and sodium hydroxide buffers were purchased from Hyclone (Logan, Utah). Tris base for pH adjustment of the PAP was purchased from Hyclone (Logan, Utah). MABSELECT™ resin for Protein A affinity chromatography experiments was purchased from GE Healthcare. Depth filtered centrate was obtained and was used as the feed stock for the Protein A affinity chromatography experiments. A Thermomixer R (Eppendorf) and two temperature controlled rooms were used to control the PAP and QPAP sample temperatures.

### Experiment 1A:

A column (1.7 cm×14.5 cm) packed with MABSELECT™ resin (33.35 mL) was equilibrated with 5 CVs of 6 mM sodium phosphate, pH 7.2 (PBS) at 6.8 mL/min (5 minute residence time). Depth filtered centrate was loaded at 27 g mAb per liter of resin using a flow rate of 6.8 mL/min at 17° C. After loading, the column was washed with 3 CVs of PBS followed by 4 CVs of 6 mM sodium phosphate pH 7.2. The product was eluted with 0.1M sodium citrate pH 3.5 for 0.5-3.0 CVs at 8.3 mL/min (4 min residence time). After elution, the PAP (9 g/L) pool was held at 17° C. for 30 minutes at pH 3.5. After the low pH hold, a portion of the Protein A product (PAP) at pH 3.5 was placed at 4, 17, and 37° C. The remainder of the PAP stream was quenched to pH 6.1 and placed at 4, 17, and 37° C. Samples (280 μL) from pH conditions 3.5 and 6.1 were taken at various time intervals, quenched using tris base (1M, 20 μL) to pH 6 and analyzed for protein aggregate content using HPSEC. The column was regenerated with 5 CVs of 50 mM sodium hydroxide, 1M sodium chloride at 8.3 mL/min and stored in 20% ethanol in PBS.

### Experiment 1B:

This experiment used the same column, feedstock, and procedure for performing Protein A affinity chromatography as described in Experiment 1A with the exception that this step was performed at 25° C. After elution, the PAP (8 g/L, pH 3.5) was subdivided and placed at 25° C. and 30° C. Samples (280 μL) at both temperatures were taken at various time intervals, quenched using tris base (1M, 20 μL) to pH 6, and analyzed for protein aggregate content using HPSEC. The column was regenerated with 5 CVs of 50 mM sodium hydroxide, 1M sodium chloride at 8.3 mL/min and stored in 20% ethanol in PBS.

### Analysis:

All PAP or QPAP samples were analyzed for mAb monomer concentration using a POROS™ Protein A ID immunoaffinity cartridge on an Agilent 1100™ HPLC system (Agilent, Palo Alto, Calif.). Protein aggregates (dimers and higher order aggregates) in each sample were quantified using a Tosoh size exclusion column (0.78 cm ID×30 cm length) on an Agilent 1100™ HPLC system. A pH probe (±0.1 pH unit accuracy) and a meter with temperature compensation (both from Fisher Scientific) were used to measure the solution pH.

### Results and Discussion

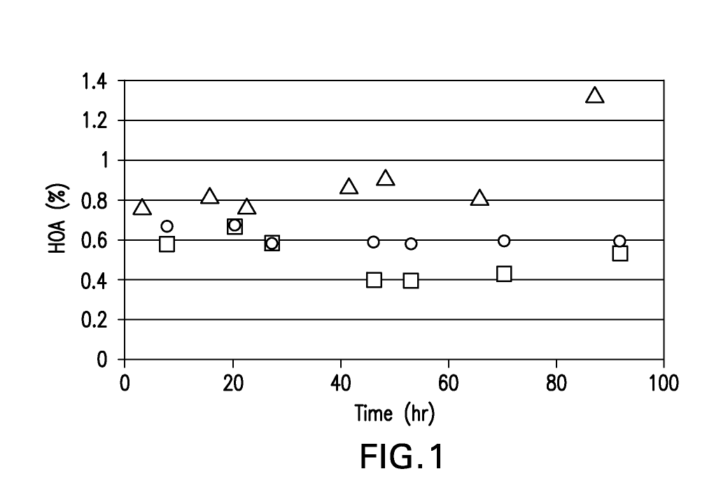

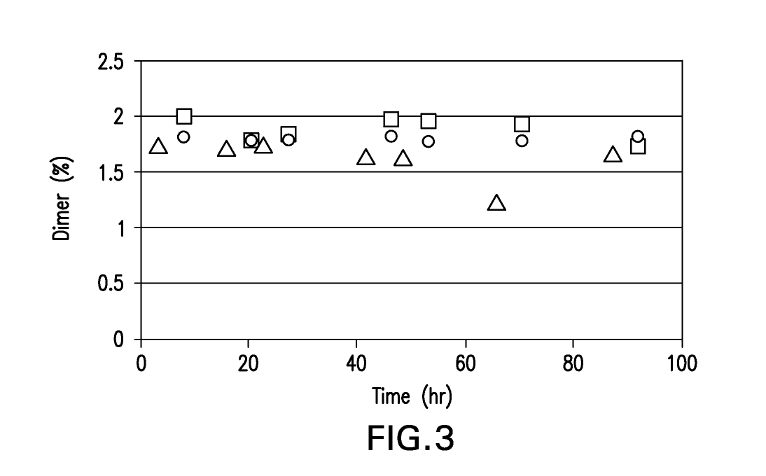

An anti-DKK-1 antibody was purified using Protein A affinity chromatography and held at various pH (3.5, 6.1) and temperature (4° C.-37° C.) values to determine the effect of pH and temperature on protein aggregation. Protein aggregation did not occur when the PAP stream was quenched to pH 6.1 after product elution from the Protein A affinity column for up to 3.5 days of hold time at 37° C., as determined by HPSEC analysis (FIG. 1). At 3.5 days, the higher order aggregate level increased from 0.8% to 1.3% while the antibody level decreased from 98% to 97% at pH 6.1 and 37° C. The dimer remained at a constant level at all temperatures at pH 6.1 (FIG. 3). The monomer remained stable at 4° C. and 17° C. at pH 6.1 (See, FIGS. 1 and 3).

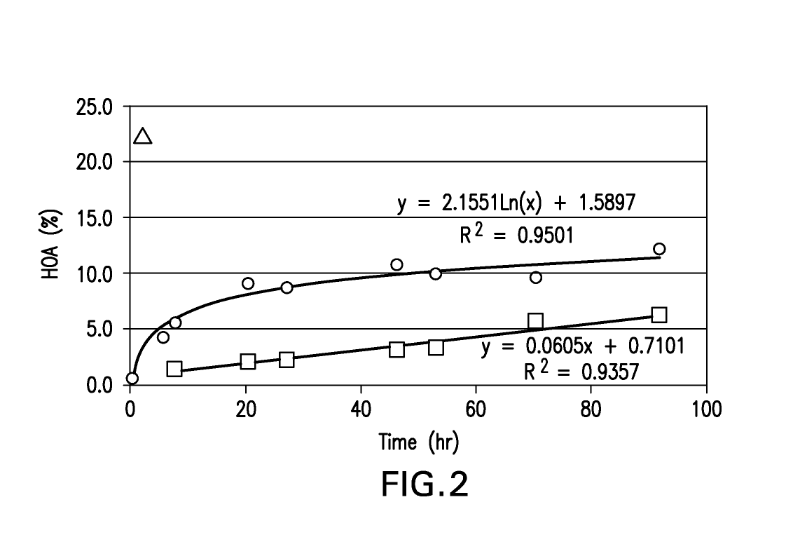

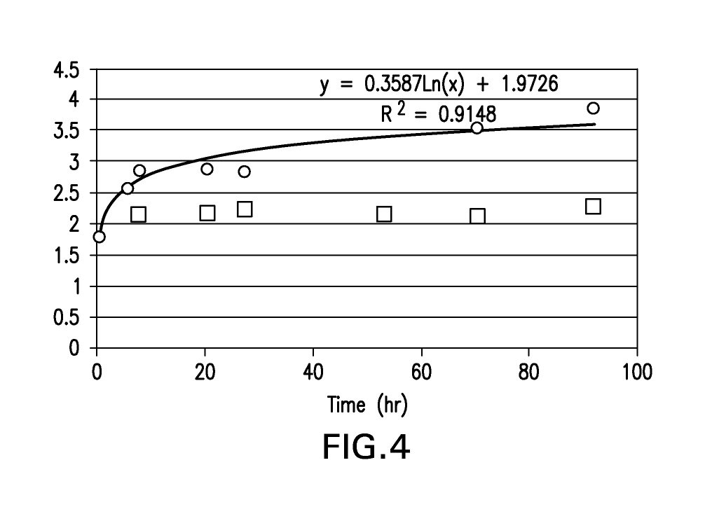

At pH 3.5, the level of higher order aggregates (HOA) significantly increased with increasing temperature (FIG. 2). In addition, the level of dimer increased by 0.8% at pH 3.6 with increasing temperature up to 17° C. (FIG. 4). At 37° C. and pH 3.6, the stability of the monomer decreases rapidly (See, FIGS. 2 and 4), which promotes significant precipitation of protein aggregates. This precipitation could affect the accuracy of the quantification of protein aggregate levels by HPSEC and represents a limitation of this method. When the temperature is lowered to 4° C., the HOA level was only 0.7% after 30 minutes and 1.5% after 8 hours (FIG. 2). Therefore, by lowering the temperature of the PAP from 17° C. to 4° C., the HOA level was significantly reduced by 4%-7%.

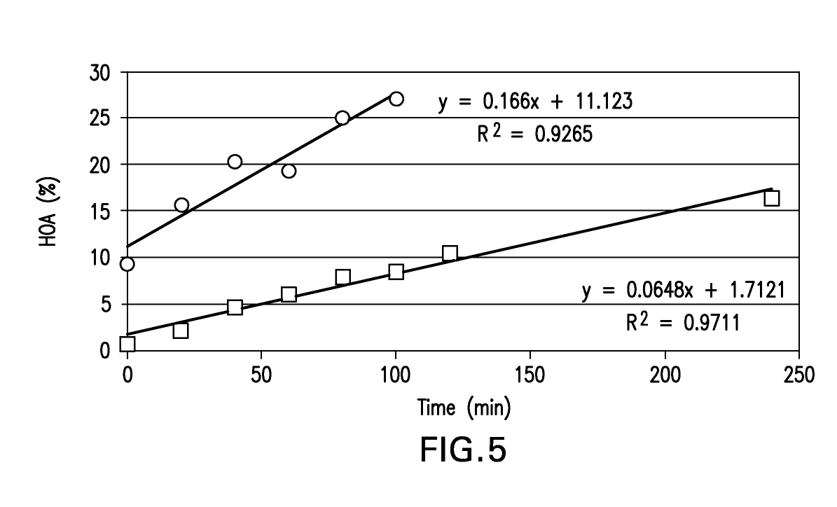

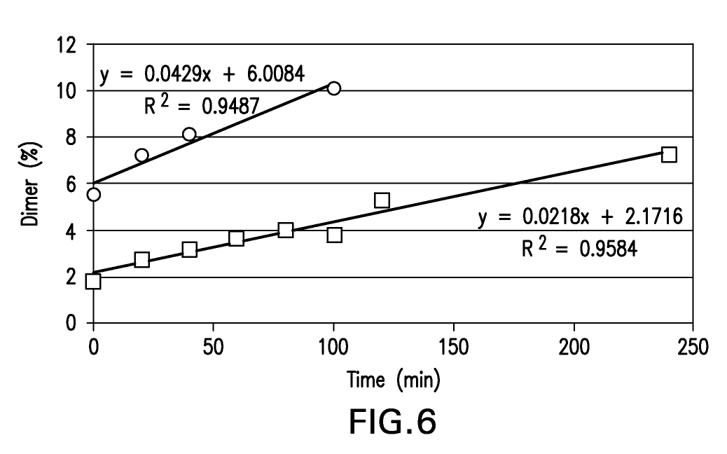

An additional experiment was performed at 25° C. and 30° C. to quantify the effect of higher temperature (>17° C.) on protein aggregate formation. The level of higher order aggregates (HOA) in the PAP (pH 3.5, 9 mg/mL anti-DKK-1 antibody) increased by 1.5%-3.0% every 20 minutes at 25° C. and by 4%-6% every 20 minutes at 30° C. (FIG. 5). The level of dimer increased up to 7% over 4 hours at 25° C. and to 10% over 1.5 hours at 30° C. (FIG. 6). In addition, the time zero sample (after product elution and collection) for the 25° C. experiment contained 0.6% HOA. Accordingly, the HOA did not form while the product was bound or eluting from the column for this experiment.

### EXAMPLE 2

### Increasing the pH of the PAP Pool for Reducing Levels of Protein Aggregates During Protein A Affinity Chromatography Elution and Subsequent Low pH Hold

To characterize the impact of pH on protein aggregation, the effect of pH (3.5-5.0) on the QPAP elution pool was evaluated with respect to an anti-DKK1 monoclonal antibody. The same procedure can be utilized with other mAbs such as anti-ADDL monoclonal antibodies.

### Materials and Methods

Small-Scale: All small-scale experiments were performed using an AKTA EXPLORER 100™ (GE Healthcare). Phosphate, citrate, and sodium hydroxide buffers were purchased from Hyclone (Logan, Utah). Tris base for pH adjustment of the PAP was purchased from Hyclone (Logan, Utah). MABSELECT™ resin for Protein A affinity chromatography experiments was purchased from GE Healthcare. Depth filtered centrate was used as the feed stock for the Protein A affinity chromatography experiments. A. Thermomixer R (Eppendorf) and two temperature controlled rooms were used to control the PAP and QPAP sample temperatures.

### Experiment 2A

A column (1.7 cm×14.5 cm) packed with MABSELECT™ resin (33.35 mLs) was equilibrated with 5 CVs of 6 mM sodium phosphate pH 7.2 (PBS) at 6.8 mL/min (5 minute residence time). Depth filtered centrate was loaded at 25 g mAb per liter of resin using a flow rate of 6.8 mL/min at room temperature (21 ° C.). After loading, the column was washed with 3 CVs of PBS followed by 4 CVs of 6 mM sodium phosphate pH 7.2. The product was eluted with 0.1 M sodium citrate pH 3.5 for 0.5-3.0 CVs at 8.3 mL/min (4 min residence time). After elution, the PAP (6.9 g/L) pool was quenched using 1M tris base (16 v %) to pH 6.

### Experiment 2B

This QPAP stream served as the feed for the pH experiment. Citrate solution (4 M, 50 to 100 μL) was added to the QPAP (20 mLs) until the solution pH reached 5.0. A sample of this solution (2 mLs) was taken at pH 5.0 and placed at 21° C. and 30° C. This procedure was repeated to generate solution conditions at pH 4.5 and 4.0. Samples (100 μL) were taken at various time points, quenched using tris base (0.5-1M, 10-30 μL) to pH 6, and analyzed for protein aggregate content using HPSEC.

### Analysis:

All PAP or QPAP samples were analyzed for mAb concentration using a POROS™ Protein A ID immunoaffinity cartridge on an Agilent 1100™ HPLC system (Agilent, Palo Alto, Calif.). Protein aggregates, i.e. dimer and higher order aggregates, in each sample were quantified using a Tosoh size exclusion column (0.78 cm ID×30 cm length) on an Agilent 1100™ HPLC system. A pH probe (Fisher Scientific) with a +/−0.1 pH unit accuracy and meter (Fisher Scientific) with temperature compensation was utilized to measure solution pH.

### Results and Discussion

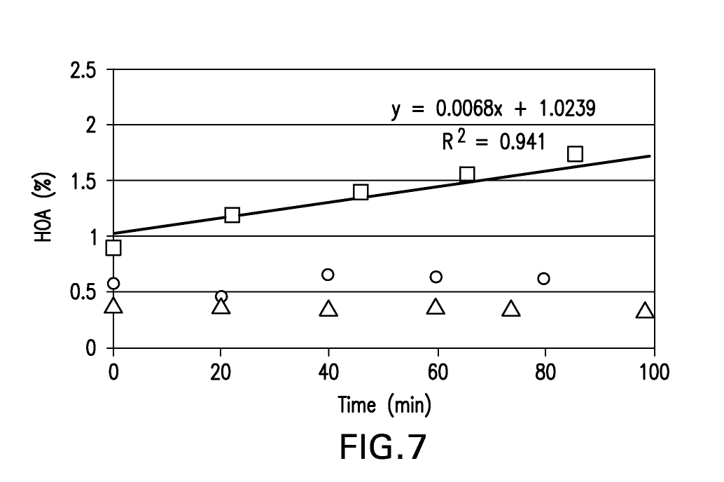

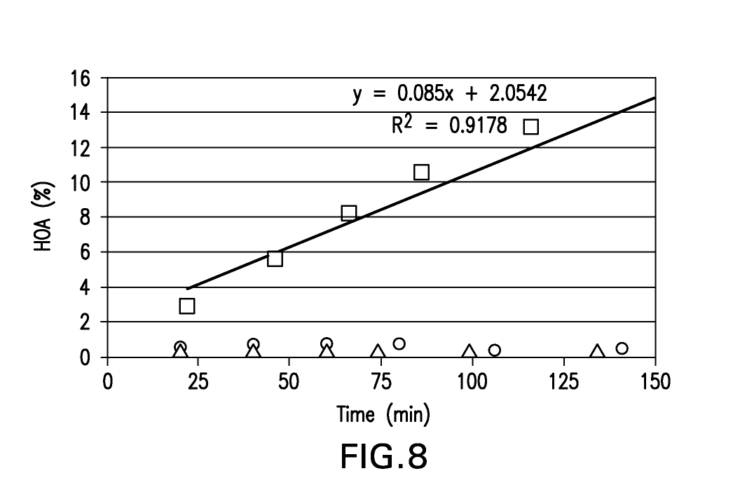

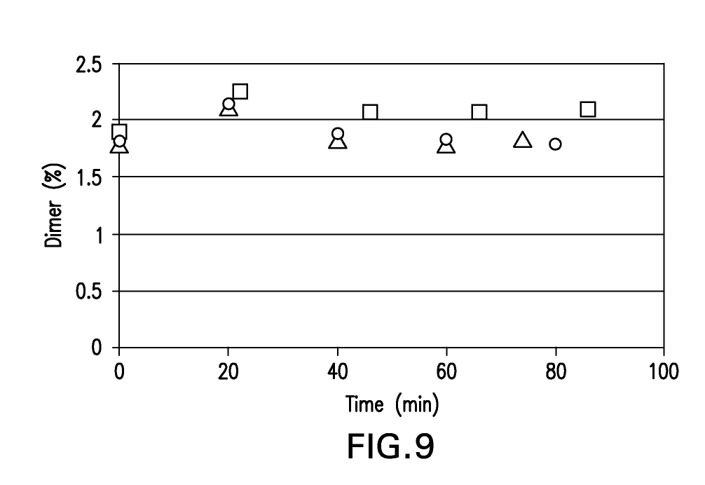

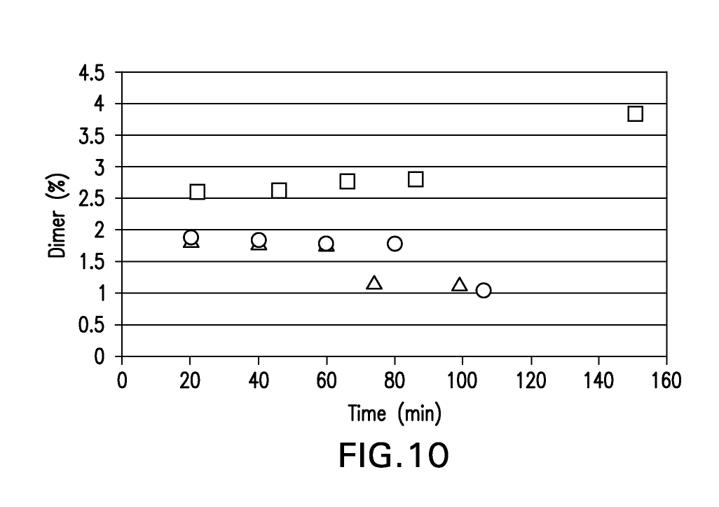

The pH of the QPAP was decreased to between pH 4.0 and pH 5.0 to determine the effect of pH on protein aggregation. The monomer was stable at 21° C. and 30° C. at pH 4.5 or greater for at least 2.5 hours (See, FIGS. 7 and 9, and 8 and 10). The HOA levels at pH 4.0 at 21° C. ranged from 0.9%-2.0% and at 30° C. ranged from 3%-13% over 2.5 hours (FIG. 7 and FIG. 8). The HOA level at pH 4.0 increased with increasing temperature, which is the same trend discovered in Example 1 at pH 3.5. The dimer level held constant at 21° C. and pH 4.0-5.0 but increased when the temperature was increased to 30° C. at pH 4.0 (FIG. 9 and FIG. 10).

In addition to temperature, pH also affected the kinetic rate of formation of protein aggregates in the PAP pool. As the pH of the PAP pool increased, the rate of higher order aggregate and dimer significantly decreased. The impact of ionic strength change in this experiment was modulated by using a highly concentrated acid. In order to prevent protein aggregation during Protein A affinity chromatography elution and subsequent low pH hold step, the elution can be performed at a higher pH.

### EXAMPLE 3

### Decoupling the Effect of Elution Buffer Concentration and pH for Reducing Levels of Protein Aggregates During Protein A Affinity Chromatography Elution and Subsequent Low pH Hold

The impact of elution buffer pH and concentration was decoupled to characterize the impact of both parameters on protein aggregation in the PAP pool. In addition, the minimum concentration of elution buffer needed to elute the monomer from the Protein A affinity chromatography column was determined. This was evaluated with respect to an anti-DKK1 monoclonal antibody.

### Materials and Methods

Small-Scale: All small-scale experiments were performed using an AKTA EXPLORER 100™ (GE Healthcare). Phosphate, citrate, and sodium hydroxide buffers were purchased from Hyclone (Logan, Utah). Tris base for pH adjustment of the PAP was purchased from Hyclone (Logan, Utah). MABSELECT™ resin for Protein A affinity chromatography experiments was purchased from GE Healthcare. Depth filtered centrate was used as the feed stock for the Protein A affinity chromatography experiments. A Thermomixer R (Eppendorf) was used to control the PAP and QPAP sample temperatures.

### Experiment A:

A column (0.5 cm×5.4 cm) packed with MABSELECT™ resin (1.06 mLs) was equilibrated with 5 CVs of 6 mM sodium phosphate pH 7.2 (PBS) at 0.2 mL/min (5 minute residence time). Depth filtered centrate was loaded at 25 g mAb per liter of resin using a flow rate of 0.2 mL/min at room temperature (17° C.-25° C.). After loading, the column was washed with 3 CVs of P135 followed by 4 CVs of 6 mM sodium phosphate pH 7.2 at 0.2 mL/min. The product was eluted using a step gradient with 10% of 0.1M sodium citrate pH 3.5 (10 mM citrate) for at least 2.5 CVs at 0.5-1.0 mL/min (1-2 min residence time). This elution step was repeated three additional times using 20%, 30%, and 100% of 0.1M sodium citrate pH 3.5 buffer 20 mM, 30 mM and 100 mM citrate, respectively). After elution, all of the PAP streams were quenched to pH 6 using 1 M tris base. The column was regenerated with 50 mM sodium hydroxide, 1 M sodium chloride at 0.5-1.0 mL/min and stored in 20 v % ethanol in PBS.

### Experiment B:

A column (1.1 cm×10.9 cm) packed with MABSELECT™ resin (10.4 mLs) was equilibrated with 5 CVs of 6 mM sodium phosphate pH 7.2 (PBS) at 2.2 mL/min (5 minute residence time). Depth filtered centrate was loaded at 27 g mAb per liter of resin using a flow rate of 2.1 mL/min at room temperature (17° C.-20° C.). After loading, the column was washed with 3 CVs of PBS followed by 4 CVs of 6 mM sodium phosphate pH 7.2 at 2.1 mL/min. The product was eluted with a step gradient of 60% 0.1M sodium citrate pH 3.5 (60 mM citrate) or 40% 0.1M sodium citrate pH 3.0 (40 mM citrate) for 0.5-3 CVs at 2.6 mL/min (4 min residence time). After immediately elution, samples of the PAP (11 g/L) pool were placed in a thermomixer at 25° C. A time zero sample was taken immediately after elution, quenched with tris base, and placed on HPSEC for protein aggregate content analysis. Samples (200 μL) were taken at various time intervals, quenched immediately using tris base (0.5-1M, 5-10 μL) to pH 6, and analyzed for protein aggregate content using HPSEC. The Protein A affinity column was regenerated with 5 CVs of 50 mM sodium hydroxide, 1 M sodium chloride at 2.4 mL/min and stored in 20% ethanol in PBS. For the 60% citrate elution (60 mM citrate), the PAP pool was subdivided into separate 2 mL aliquots. Citrate (4M, 5-10 μL) was added to an aliquot to reach pH 3.4 or 3.6. Phosphoric acid (8 v %, 10 μL) was added to an aliquot to reach pH 3.6.

### Experiment C:

The citrate concentration was reduced to 50 mM in the Protein A elution buffer to determine if acid concentration has an impact on anti-DKK-1 stability. This reduced acid concentration impacted the gradient of the pH slope during elution, which resulted in a higher PAP pool pH of 3.9 versus 3.6.

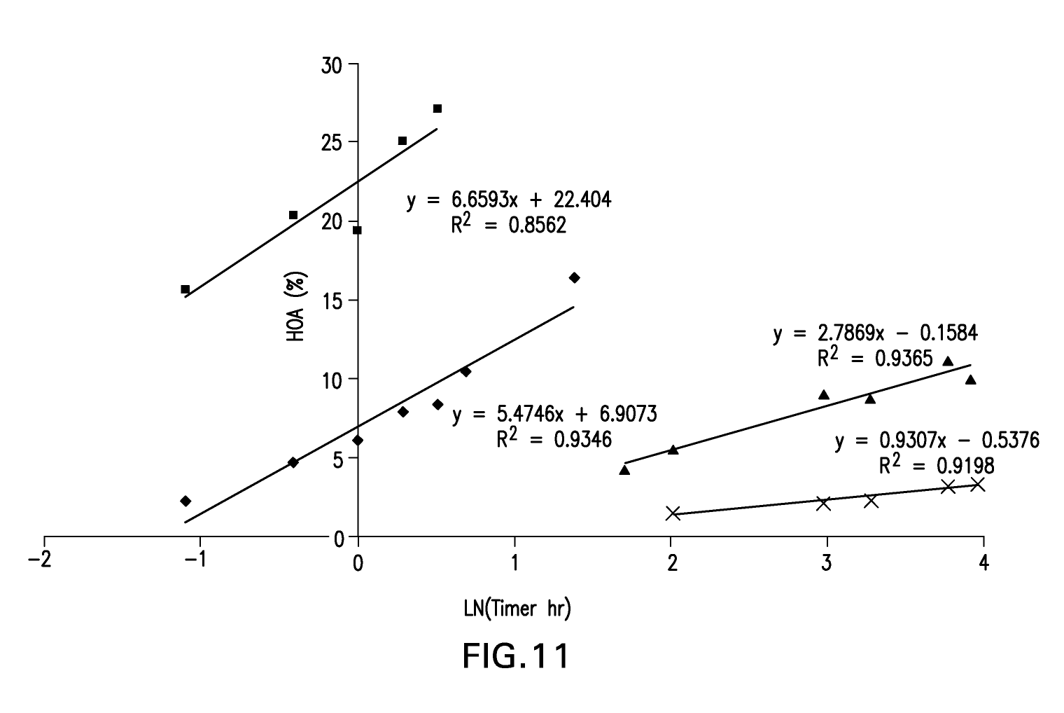

Depth filtered centrate (1.7 g/L anti-DKK-1 mAb) was loaded onto a Protein A column (V=33.35 mLs, Plates: 2026 N/m², Asymmetry: 1.33) using a residence time of 5 minutes at 23-25° C. After loading, the column was washed with 3 CVs of PBS followed by 4 CVs of 6 mM sodium phosphate pH 7.2 at 0.2 mL/min. For the elution, a 50% gradient of 100 mM citrate pH 3.5 was used to elute the anti-DKK-1 monoclonal antibody from the resin. During elution, fractions were collected every 0.5 CV from 0.5 to 3.0 CVs. These fractions were analyzed for monomoer concentration, pH, and conductivity and then pooled for a final concentration of 7.2 g/L. Samples of the PAP stream were placed at various temperatures (4, 15, 20, and 23-25° C.) and pH values (3.9, 4.2, 4.5, 5.0) and analyzed at various time points for aggregation using HPSEC (FIG. 11).

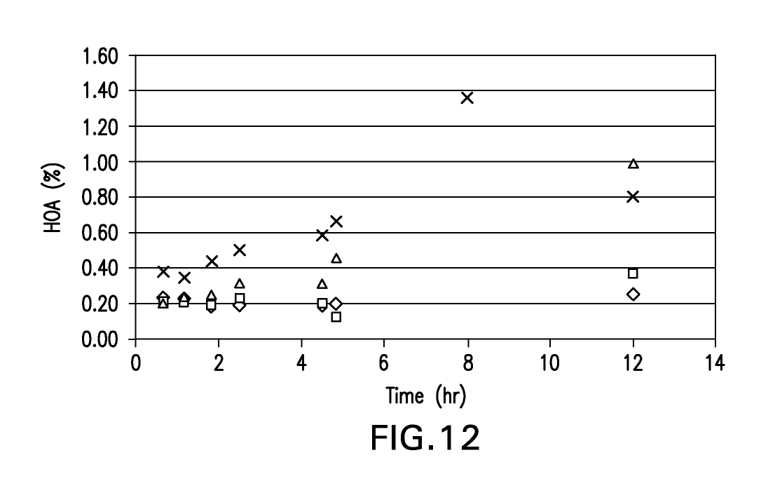

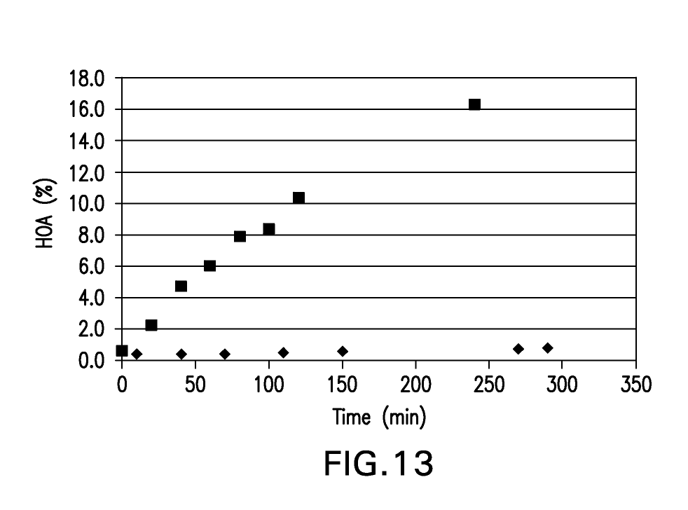

The pH gradient changed slightly when the acid concentration was reduced to 50 mM, which resulted in a PAP pH of 3.9 versus 3.6 for 100 mM citrate. However, the elution profiles overlapped and the product collection window will remain the same. The Protein A yield for the 50% elution was 94%. The HOA profile using the 50 mM citrate elution at the various temperatures above is shown in FIG. 12. A comparison between the 50 mM and 100 mM citrate elutions at 23-25° C. is represented in FIG. 13.

### Analysis:

All PAP samples were analyzed for mAb concentration using a POROS™ Protein A ID immunoaffinity cartridge on an Agilent 1100™ HPLC system (Agilent, Palo Alto, Calif.). Protein aggregates, i.e. dimer and higher order aggregates, in each sample were quantified using a Tosoh size exclusion column (0.78 cm ID×30 cm length) on an Agilent 1100™ HPLC system. A pH probe (Fisher Scientific) with a ±0.1 pH unit accuracy and meter (Fisher Scientific) with temperature compensation was utilized to measure solution pH.

### Results and Discussion

The Protein A affinity column was eluted with various citrate concentrations to determine the lowest concentration of citrate possible for elution of the monomer from the resin. The 10 v % and 20 v % citrate concentrations eluted 80% of the monomer that was bound to the column (not shown). When the citrate concentration was increased to 30 v %, only 2% additional monomer eluted from the column. Therefore, the minimum concentration to elute ≧80% monomer from the column was 20 mM citrate.

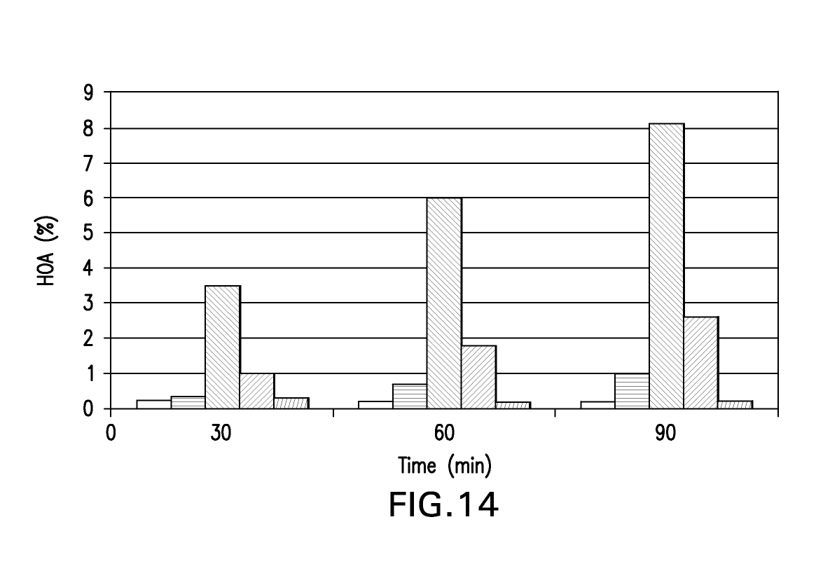

Different citrate concentrations were tested to determine the impact of citrate concentration on monomer stability at various pH points. When the citrate concentration was lowered to 60 mM and pH was increased to 3.8, the HOA level was 0.3% versus 4%-5% for 100 mM citrate (pH 3.5) at 25° C. (FIG. 14). As the citrate concentration was reduced to 40 mM citrate at pH 3.6, the HOA level remained at 0.3% for at least 3 hours. Therefore, the monomer stability was a function of citrate concentration as well as pH. When phosphoric acid was added instead of citrate to pH adjust the elution pool, the HOA level increased by approximately 0.3% for every time point sample up to 2 hours. Therefore, the addition of phosphoric acid increased the rate of HOA formation faster than citrate at the same solution pH (3.6).

At the PAP pool pH 3.6, the following four different acid concentrations were tested: 40 mM, 75 mM, and 100 mM citrate, and 60 mM citrate with 0.1 v % phosphoric acid. The level of HOA increased as the citrate concentration increased. In addition, the level of HOA increased significantly over time at citrate concentrations greater than 75 mM. For example, within a one hour time frame, the rate of HOA in 100 mM citrate was 2% per 20 minutes compared to 0.2%-0.3% per 20 minutes in 75 mM citrate at the same pH of 3.6. The addition of phosphoric acid to the PAP pool increased the rate of HOA formation than citrate at the same solution pH of 3.6. The concentration of citrate in the PAP pool had an impact on HOA formation when the pH was held constant.

From FIG. 12 above, the PAP was extremely stable and contained ≦0.4% HOA at ≦15° C. over at least 12 hours with the 50 mM citrate elution condition at pH 3.9. The anti-DKK-1 mAb also showed improved stability at higher temperatures in the PAP. For example, after a 12 hour hold at 20-25° C., the PAP contained 1.0%-1.4% HOA. The HOA level in the PAP for the low pH hold time of 30-60 minutes was 0.2%-0.4% at 20-25° C., which is below the 3%-6% HOA level in the lot as shown by the comparison in FIG. 13. The total amount of protein degradation (HOA plus dimer) in the 50 mM citrate PAP was 1.5%, which met the goal of ≦5%. Therefore, reducing the citrate concentration and increasing the pH was proven to significantly reduce the HOA level in the PAP from 3%-6% to 0.5% for a hold time of ≦60 minutes.

### EXAMPLE 4

### Impact of pH and Citrate Concentration on the Thermal-Induced Unfolding of a Monoclonal Antibody as Measured by Differential Scanning Calorimetry

Differential Scanning Calorimetry is a tool used to measure protein stability. Protein stability is largely dependent on the environment, which has the ability to both stabilize and destabilize the folded structure of the protein. DSC operates by measuring the heat capacity of a protein solution during a temperature ramp as compared to the heat capacity of a solvent reference. The differential heat capacity between the protein solution and the solvent reference provides a profile representing the denaturation of the protein. From this profile, the apparent melting temperature I can be determined. The denaturation of a protein into an unfolded state often results in undesirable events, such as aggregation or chemical degradation (19, 20, 21, 22, 23).

Differential scanning calorimetry (DSC) provides some insight into the mechanisms of folding by measuring the temperature-induced unfolding of proteins. The information provided by DSC is useful in a variety of applications involving protein stability, such as clone selection, formulation development, and protein characterization (24, 25). Having a relatively easy method to identify the most stable drug compound early in the development process provides a huge advantage by potentially shortening the time-to-market. DSC is also critical in formulation development. Changes in protein environment, such as pH, ionic strength, and other excipients can impact the folded structure of the protein, resulting in a shift in the melting temperature. By screening various additives in the formulation buffer, DSC provides a quick way to optimize buffer composition.

Aggregation of therapeutic proteins has very serious implications, ranging from difficulties in purification processing to immunogenic responses in vivo. The denaturation, and subsequent aggregation, of proteins is sensitive to many things, including pH and temperature. Thus, it is critical to the stability of therapeutic proteins to regulate both of these factors.

Monoclonal antibodies have multiple domains, each of which is impacted differently by pH and ionic strength. DSC was used to evaluate the effect of ionic strength (citrate concentrations of 30 mM, 60 mM, and 100 mM) and pH (3.0 to 6.0) on the protein's thermal stability.

### Materials and Methods

Solutions were prepared at a concentration of 30 mM, 60 mM and 100 mM citric acid (Sigma, St. Louis). The citrate solutions were pH adjusted to target values of 3.0, 3.5, 4.0, 4.5, 5.0, 5.5, and 6.0 pH units using sodium hydroxide (Fisher Chemicals). The pH measurements were obtained using an Accumet pH meter (Fisher Scientific).

An anti-DKK-1 antibody at 54.2 mg/mL in formulation buffer was diluted to 1 mg/mL with the citrate buffers at each pH. The concentration and pH of the final 1 mg/mL solutions were measured. The formulation buffer was diluted in the same ratio for use as the reference buffer.

Differential scanning calorimetry (DSC) was conducted on a MicroCal™ VP-DSC. The temperature was ramped from 25° C. to 95° C. at a rate of 1° C./min. The raw DSC profiles were analyzed by subtracting the reference buffer, normalizing the concentrations, and performing baseline correction using Origin software. Samples were run in duplicate.

### Results and Discussion

The impact of pH and citrate concentration on the thermal stability of an anti-DKK1 antibody was examined using DSC. The results indicate that the melting temperature is decreased as the pH is decreased. The results also suggest that citrate has a larger impact on the thermal stability of this monoclonal antibody at low pH values.

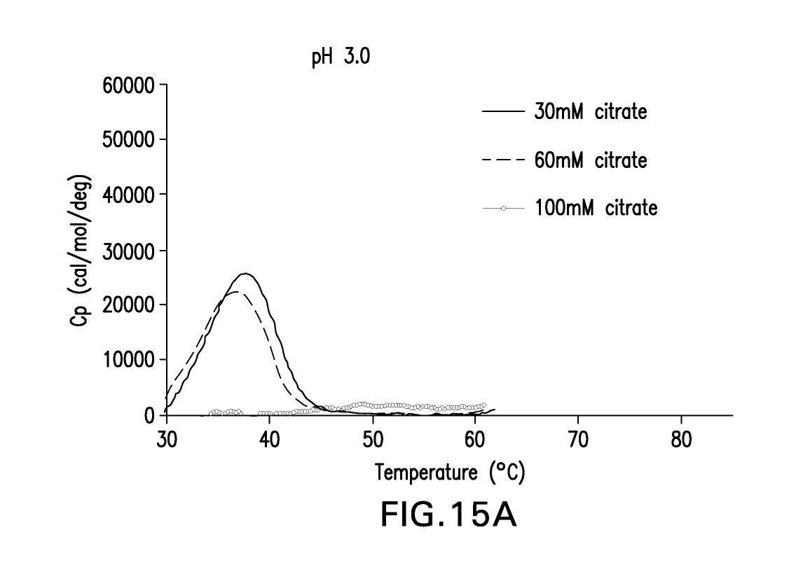

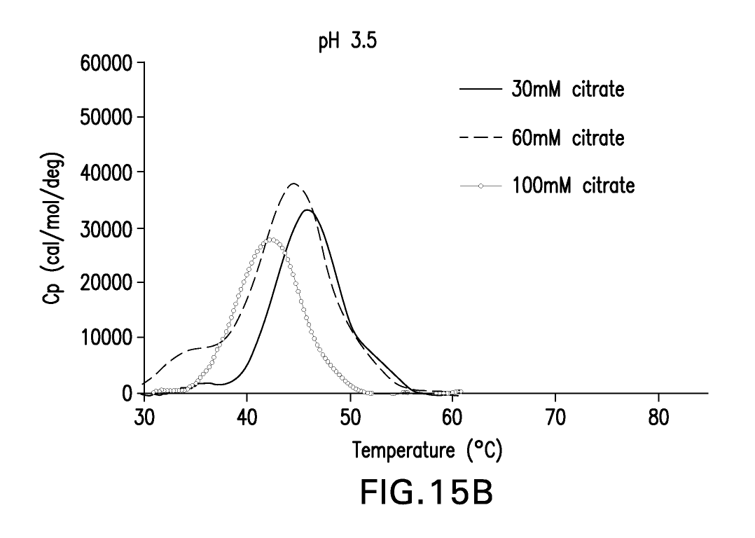

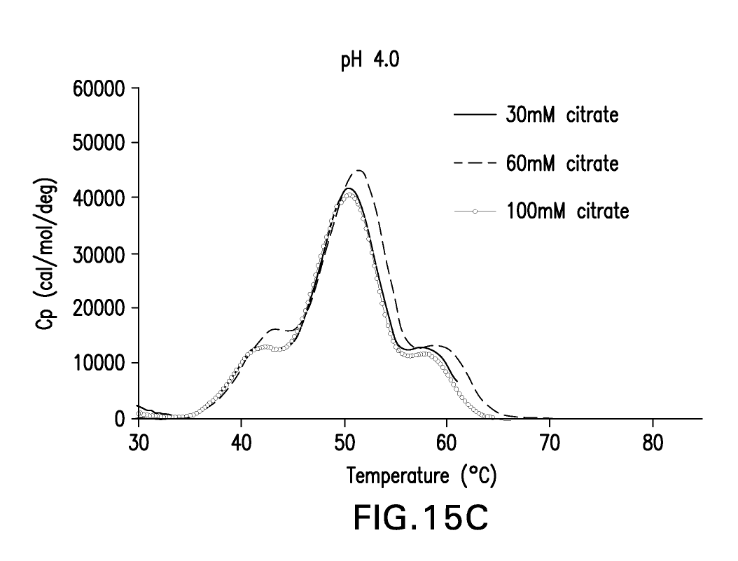

At pH values below 4.5, the anti-DKK-1 monoclonal antibody diluted with citrate buffers is more thermally stable in low citrate concentrations (FIG. 15A, B, C). At pH 3.0, it appears that 100 mM citrate has completely unfolded the protein and transitions are not present in the profile. For 30 mM and 60 mM citrate, a single transition is apparent, suggesting two of the antibody's domains are unfolded. The data supports that this single transition represents the unfolding of the Fab region of the antibody. As the pH increases to 4.0, the unfolding temperature of the domains increased and three transitions were found. The least stable transition is most likely the unfolding of the C_{H}2 domain, followed by the Fab fragment and the C_{H}3 domain.

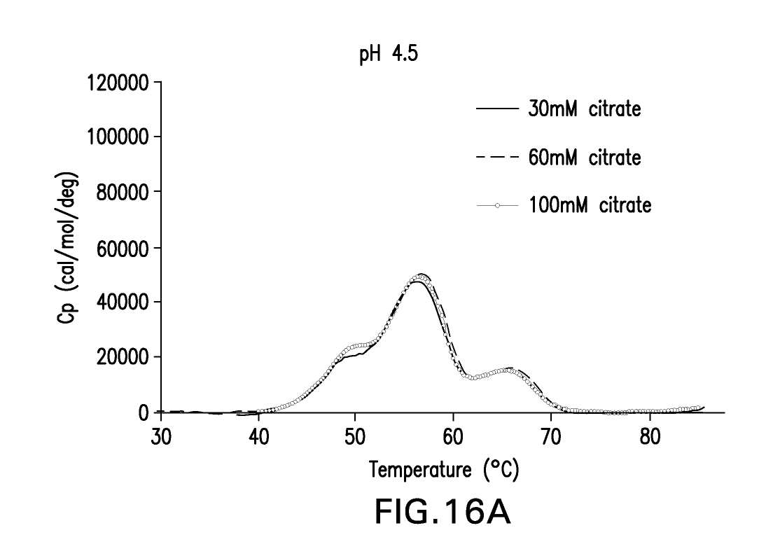

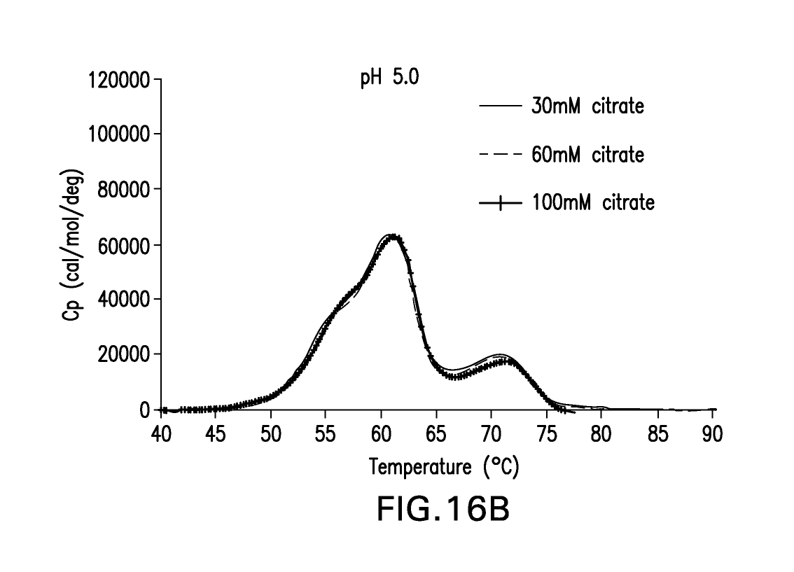

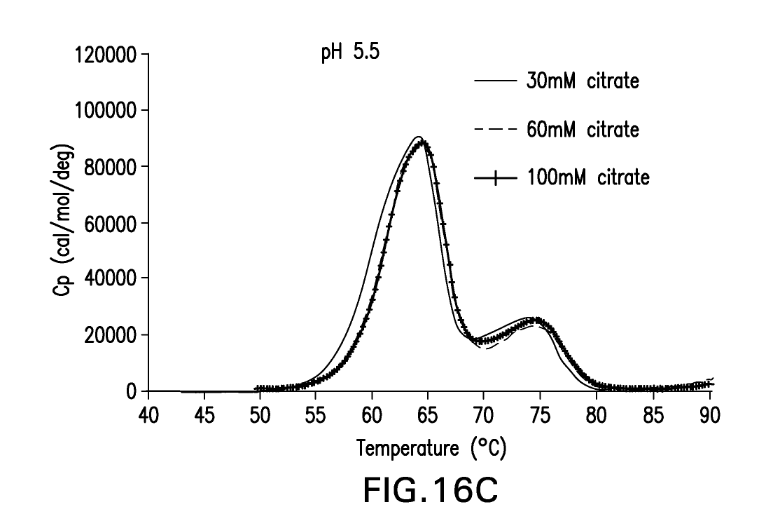

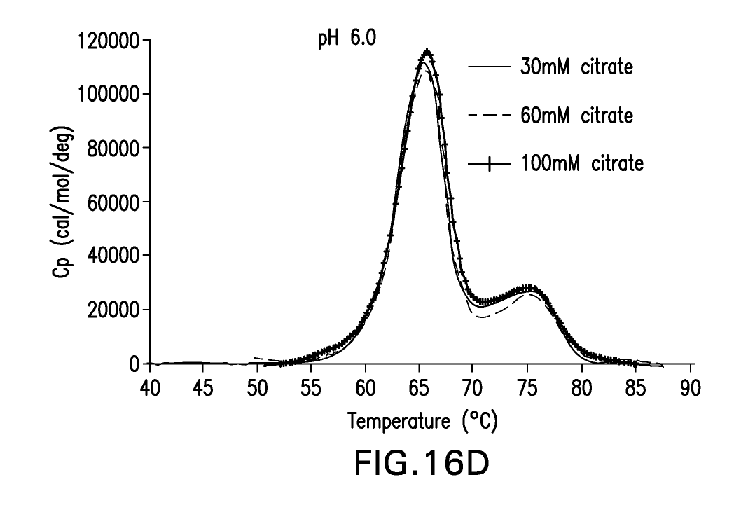

At pH values of 4.5 and above, the apparent transition temperatures for the anti-DKK-1 monoclonal antibody are independent of citrate concentration (FIG. 16A, B, C, D). The melting temperatures for the antibody increase as pH increases. At higher pH values, two domains unfold simultaneously, resulting in a profile that has a large enthalpy for one transition and a second transition with a lower enthalpy. We conclude from results with other monoclonal antibodies that the C_{H}2 domain and Fab have similar melting temperatures at high pH values, corresponding to the first large transition. The unfolding of the C_{H}3 domain has a higher melting temperature and corresponds to the second transition.

Citrate concentration and pH impact the thermal-induced unfolding of monoclonal antibodies. As the pH was lowered, the thermal stability of an anti-DKK-1 monoclonal antibody decreases. Citrate concentration only impacts apparent transition temperatures at low pH values. Lower concentrations of citrate result in higher melting temperatures.

### EXAMPLE 5

### The Effect of Amino Acid Stabilizers on Protein Aggregation

Acidic conditions are required in order to elute a protein or antibody from the Protein A affinity resin. Exposure to these acidic conditions at low pH (3-4) can result in the formation of protein aggregates. The addition of a stabilizer to the Protein A elution buffer has been shown to increase the stability of a protein at low pH. Arginine was selected as the stabilizer for this experiment to determine if the presence of arginine in the elution buffer will decrease level of higher order aggregates and subsequently increase mAb stability. All experiments were performed at 25° C.

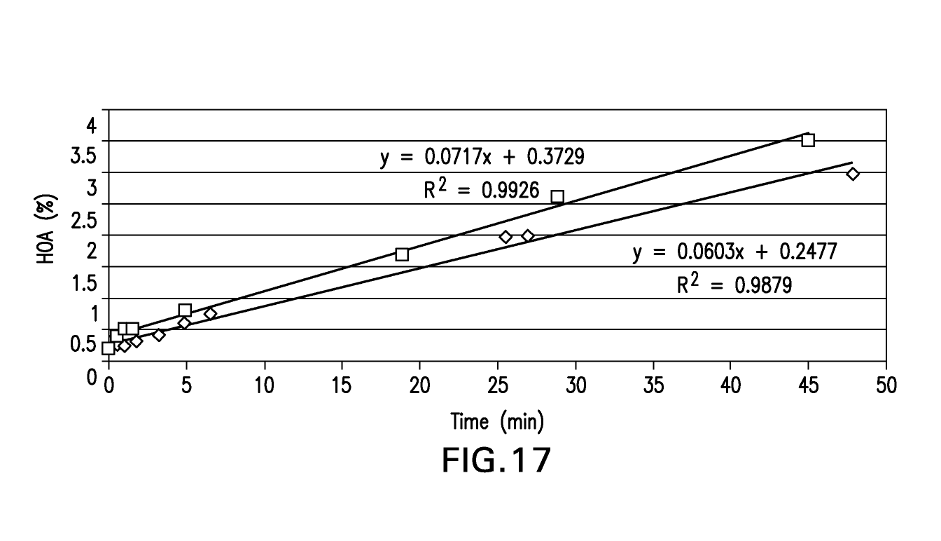

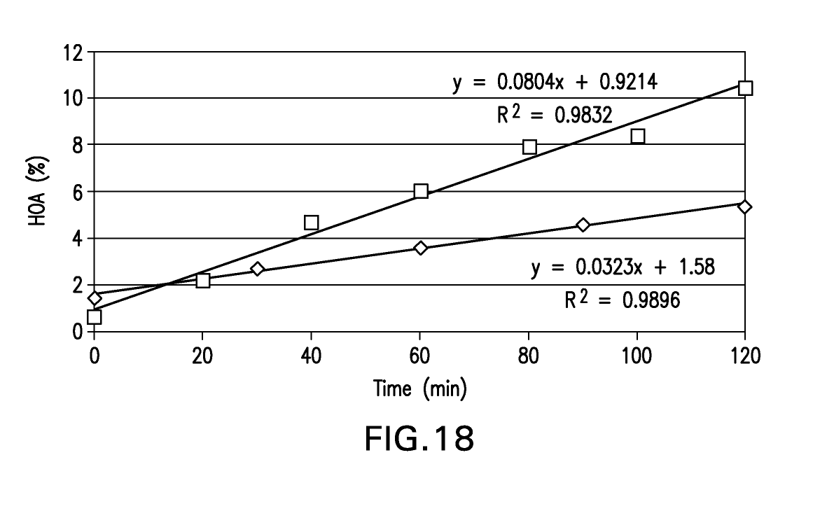

For an elution using 60 mM citrate with and without arginine (50 mM), there was only a 0.01% decrease in HOA percent per hour compared to the control (FIG. 17). However, for an elution using 100 mM citrate with and without arginine (250 mM), there was a 2.9% decrease in higher order aggregate percent per hour compared to the control (FIG. 18). Therefore, arginine was effective at decreasing the levels of higher order aggregates in a 100 mM citrate solution and can be used as another option to decrease levels of aggregates during the Protein A affinity chromatography and subsequent low pH hold steps.

### EXAMPLE 6

### Investigating the Effect of Protein Concentration on Monomer Stability in Citrate and Acetate Elution Buffers Used in Protein A Affinity Chromatography

The impact of protein concentration in citrate and acetate buffers was investigated to determine the effect of concentration on the aggregation rate in each buffer system. In addition, the citrate and acetate buffers at pH 4.0 were compared in order to determine the effect of the acid type on aggregation.

### Materials and Methods

Small-Scale: All small-scale experiments were performed using an AKTA EXPLORER 100™ (GE Healthcare). Sodium phosphate buffer was purchased from Hyclone (Logan, Utah). Acetate and citrate were purchased from Fisher Scientific (Pittsburgh, Pa.). Tris base for pH adjustment of the PAP was purchased from Hyclone (Logan, Utah). MABSELECT™ resin for Protein A affinity chromatography experiments was purchased from GE Healthcare. Quenched protein A product was used as the feed stock for these experiments. A Thermomixer R (Eppendorf) was used to control the PAP and QPAP sample temperatures.

The QPAP stream served as the feed for this experiment. The feed was diafiltered into four to five volumes of sodium phosphate buffer using a 30 kDa membrane at a centrifuge speed of 4500 rpm. After the diafiltration, each solution was concentrated to either 1×, 2×, and 4× of the original concentration. Half of the samples at the various concentrations were diafiltered into at least four volumes of sodium acetate (50 mM, pH 5.0) solution.

For the first set of the three different concentrated solutions taken from Example 2, citrate (15 mM) was added to each solution to reach pH 4.0. Samples (2 mL) of each solution were taken and placed at 21° C. (FIG. 7). For the second set of concentrated solution, glacial acetic acid was added to reach pH 4.0 (total 85 mM acetate). Samples (2 mL) of each solution were taken and placed at 25° C. For each set of experiments, samples (140 μL) were taken at various time points, quenched using tris base (0.25 M-1.0 M, 10-20 μL) to pH 6, and analyzed for protein aggregate content using HPSEC.

### Analysis:

Samples were analyzed for monomeric mAb concentration using a POROS™ Protein A ID immunoaffinity cartridge on an Agilent 1100™ HPLC system (Agilent, Palo Alto, Calif.). Protein aggregates, i.e. dimer and higher order aggregates, in each sample were quantified using a Tosoh size exclusion column (0.78 cm ID×30 cm length) on an Agilent 1100™ HPLC system. A pH probe (Fisher Scientific) with a +/−0.1 pH unit accuracy and meter (Fisher Scientific) with temperature compensation was utilized to measure solution pH.

### Results and Discussion

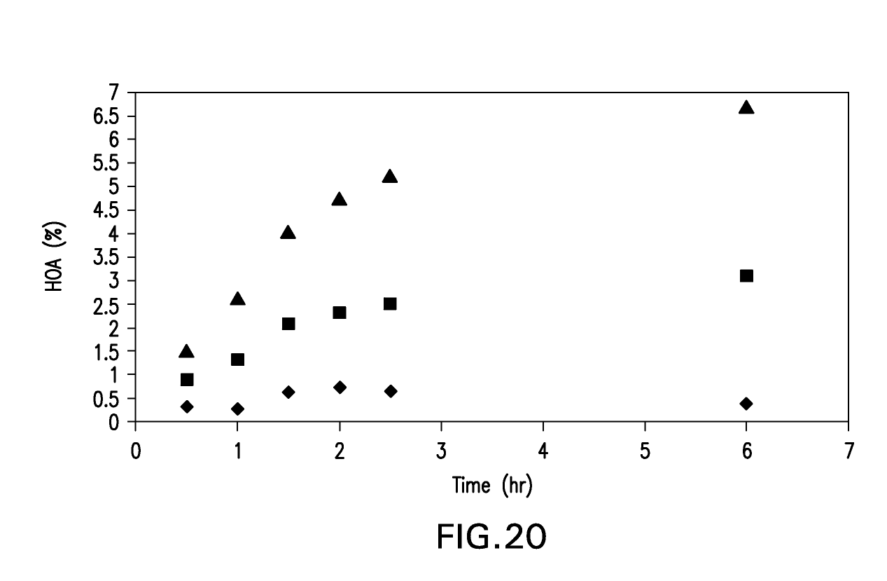

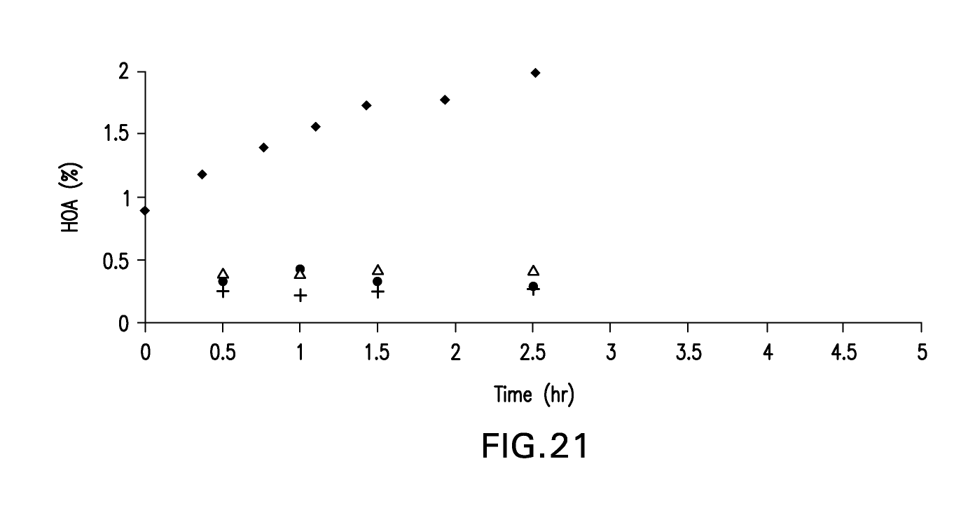

The effect of mAb concentration in citrate and acetate buffers was investigated to determine the impact of concentration on the aggregation rate in each buffer system. As the mAb concentration increased in the citrate (15 mM, pH 3.5) solution, the rate of higher order aggregate formation increased as well (FIG. 20). For example, after a one hour hold time, the level of higher order aggregates increased from 0.3% at 8 mg/mL mAb concentration to 2.6% at 34 mg/mL mAb concentration. Therefore, the concentration of protein in solution impacts the rate of higher order aggregate formation in a citrate buffered system at low pH. However, for the acetate (85 mM, pH 4.0) solution, the level of higher order aggregates remained constant at less than 1% for 5 mg/mL, 11 mg/mL, and 37 mg/mL mAb concentrations (FIG. 21).

To determine the impact of acid type on higher order aggregate formation, the results from this acetate experiment at pH 4.0 at 25° C. were graphed with the results from the citrate experiment at pH 4.0 st 21° C. (FIG. 21). At pH 4.0, the rate of higher order aggregates starts to increase by 0.2% every 30 minutes in a citrate buffered system while the level of higher order aggregates remains constant in the acetate buffered system. Therefore, at pH 4.0, the mAb stability was greater in acetate than citrate buffer.

### Conclusions

In addition to temperature and pH, protein concentration also affected the rate of protein aggregate formation in the PAP pool. As the mAb concentration was increased in a citrate buffered solution at pH 3.5, the rate of higher order aggregate significantly increased. However, the level of higher order aggregates remained less than 1% in an acetate buffered solution at pH 4.0 for various protein concentrations, which ranged from 5 mg/mL to 37 mg/mL. When the acetate and citrate buffer systems are compared at pH 4.0 with a protein concentration of 5 mg/mL to 11 mg/mL, the mAb contained greater stability in the acetate buffer. Therefore, the type of elution buffer played a role in mAb stability with acetate being more stable than citrate buffer at pH 4.0. Acetate can be used as an alternative buffer for mAb elution from a Protein A affinity column.

### EXAMPLE 7

### Investigating the Effect of pH, Temperature, Buffer Concentration and Protein Loading on the Level of Protein Aggregates During Protein A Affinity Chromatography Elution and Subsequent Low pH Hold

### Materials and Methods:

Small-Scale: All small-scale experiments were performed using an AKTA EXPLORER 100™. Phosphate, citrate, and sodium hydroxide buffers were purchased from Hyclone (Logan, Utah). Tris base for pH adjustment of the PAP was purchased from Hyclone (Logan, Utah). MABSELECT™ resin for Protein A affinity chromatography experiments was purchased from GE Healthcare. Depth filtered centrate was obtained and was used as the feed stock for the Protein A affinity chromatography experiments. A Thermomixer R (Eppendorf) and two temperature controlled rooms were used to control the PAP and QPAP sample temperatures.

### Experiment:

A column (1.7 cm×14.5 cm) packed with MABSELECT™ resin (10 mL) was equilibrated with 5 CVs of 6 mM sodium phosphate, 100 mM NaCL, pH 7.2 buffer at 2.0 mL/min (5 minute residence time). Depth filtered centrate was loaded at the concentration listed in Table 1 (g mAb per liter of resin) using a flow rate of 2.0 mL/min at the temperature listed in Table 1. After loading, the column was washed with 3 CVs of 6 mM sodium phosphate, 100 mM NaCl, pH 7.2 buffer at 2 ml/min, which was followed by 4 CVs of 6 mM sodium phosphate, pH 72 buffer at 2 ml/min. The product was eluted with 3 CVs of elution buffer (mixture of citric acid anhydrous and trisodium citrate salt to target pH value) at 100% step gradient at 2 ml/min (start at 0.5 and end at 3.5) (4 min residence time). The elution buffers used in this experiment are as follows:

60 mM sodium citrate, pH 3.5

40 mM sodium citrate, pH 3.2

40 mM sodium citrate, pH 3.8

80 mM sodium citrate, pH 3.2

80 mM sodium citrate, pH 3.8

After elution, the product eluant sample was analyzed for protein aggregate content using HPSEC at the following time points: 0, 15, 30, 60, 120 min. Sample will be 200 microliters with 40 microliters of 0.25M tris base added to quench the sample. The column was regenerated with 5 CVs of 50 mM sodium hydroxide, 1M sodium chloride buffer at 2.0 mL/min and stored in 20 v % ethanol solution in PBS.

### Analysis:

All samples were analyzed for mAb monomer concentration using a POROS™ Protein A ID immunoaffinity cartridge on an Agilent 1100™ HPLC system (Agilent, Palo Alto, Calif.). Protein aggregates (dimers and higher order aggregates) in each sample were quantified using a Tosoh size exclusion column (0.78 cm ID×30 cm length) on an Agilent 1100™ HPLC system. A pH probe (±0.1 pH unit accuracy) and a meter with temperature compensation (both from Fisher Scientific) were used to measure the solution pH.

### Results:

The effect of pH, temperature, buffer concentration and protein loading was investigated to determine their impact on yield and protein aggregation. The results of the above experiment can be found in Table 1 below.

At 10 degrees C. and pH 3.2, loading or citrate concentration does not significantly impact yield or aggregation levels. At 10 degrees C. and pH 3.8, loading and/or citrate concentration decreases yield but aggregates remain at low levels (<5%). At 30 degrees C. and pH 3.2, loading does not significantly impact aggregation but temperature has a huge effect on decreasing yield and increasing aggregation levels for 80 mM citrate concentration but little effect is seen in 40mM citrate. As the pH changes from 3.2 to 3.8 at 30C, the aggregate levels decrease significantly and yield increases at 40 to 80 mM citrate.

TABLE 1
Aggregation data for anti-DKK-1 mAb at various concentrations,
pH, protein loading, and temperatures

| | HOA | Dimer | anti-DKK-1 |
|---|---|---|---|
| | (%) | (%) | mAb (%) |
| Sample | | | |
| anti-DKK-1 mAb | 4.6 | 2.4 | 93 |
| std, 10x dil | | | |
| Time (min) | | | |

80 mM, pH 3.2, 20 g/L, 10 C.

| | | | |
|---|---|---|---|
| 0 | 0.6 | 1.6 | 97.8 |
| 15 | 0.5 | 1.5 | 98.0 |
| 30 | 0.6 | 1.6 | 97.8 |
| 60 | 0.6 | 1.6 | 97.8 |
| 120 | 0.7 | 1.7 | 97.5 |

80 mM, pH 3.2, 40 g/L, 10 C.

| | | | |
|---|---|---|---|
| 0 | 0.5 | 1.6 | 98.0 |
| 15 | 0.5 | 1.6 | 97.9 |
| 30 | 0.5 | 1.6 | 97.9 |
| 60 | 0.7 | 1.6 | 97.7 |
| 120 | 0.9 | 1.7 | 97.5 |

40 mM, pH 3.2, 20 g/L, 10 C.

| | | | |
|---|---|---|---|
| 0 | 0.6 | 1.4 | 98.0 |
| 15 | 0.5 | 1.4 | 98.0 |
| 30 | 0.5 | 1.4 | 98.0 |
| 60 | 0.5 | 1.4 | 98.1 |
| 120 | 0.6 | 1.4 | 98.1 |

40 mM, pH 3.2, 40 g/L, 10 C.

| | | | |
|---|---|---|---|
| 0 | 0.5 | 1.5 | 98.0 |
| 15 | 0.5 | 1.5 | 98.0 |
| 30 | 0.5 | 1.6 | 97.9 |
| 70 | 0.5 | 1.6 | 97.9 |
| 120 | 0.5 | 1.5 | 98.0 |

40 mM, pH 3.8, 40 g/L, 10 C.

| | | | |
|---|---|---|---|
| 0 | 0.4 | 0.9 | 98.7 |
| 15 | 0.4 | 0.9 | 98.6 |
| 30 | 0.4 | 0.9 | 98.7 |
| 60 | 0.4 | 0.9 | 98.7 |
| 120 | 0.4 | 0.9 | 98.7 |

40 mM, pH 3.8, 20 g/L, 10 C.

| | | | |
|---|---|---|---|
| 0 | 0.5 | 0.9 | 98.7 |
| 15 | 0.4 | 0.9 | 98.6 |
| 30 | 0.4 | 0.9 | 98.7 |
| 60 | 0.5 | 1 | 98.6 |
| 120 | | | |

80 mM, pH 3.8, 40 g/L, 10 C.

| | | | |
|---|---|---|---|
| 0 | 0.4 | 1 | 98.7 |
| 15 | 0.5 | 1.1 | 98.4 |
| 30 | 0.4 | 1 | 98.7 |
| 60 | 0.4 | 1 | 98.6 |
| 120 | 0.4 | 1 | 98.6 |

80 mM, pH 3.8, 20 g/L, 10 C.

| | | | |
|---|---|---|---|
| 0 | 0.5 | 0.9 | 98.6 |
| 15 | 0.5 | 1 | 98.5 |
| 30 | 0.5 | 0.9 | 98.6 |
| 60 | 0.5 | 1 | 98.5 |
| 120 | 0.4 | 1 | 98.6 |

60 mM, pH 3.5, 30 g/L, 20 C.

| | | | |
|---|---|---|---|
| 0 | 0.5 | 1.5 | 97.9 |
| 15 | 0.6 | 1.5 | 97.9 |
| 30 | 0.7 | 1.6 | 97.7 |
| 60 | 0.7 | 1.6 | 97.7 |
| 120 | 1 | 1.7 | 97.3 |

60 mM, pH 3.5, 30 g/L, 20 C.

| | | | |
|---|---|---|---|
| 0 | | | |
| 15 | | | |
| 30 | 0.6 | 1.6 | 97.8 |
| 60 | 0.8 | 1.7 | 97.5 |
| 130 | 0.9 | 1.7 | 97.4 |

60 mM, pH 3.5, 30 g/L, 20 C.

| | | | |
|---|---|---|---|
| 0 | 0.6 | 1.6 | 97.8 |
| 15 | 0.5 | 1.6 | 97.8 |
| 30 | 0.6 | 1.7 | 97.7 |
| 60 | 0.7 | 1.6 | 97.7 |
| 120 | 0.8 | 1.7 | 97.5 |

40 mM, pH 3.2, 40 g/L, 30 C.

| | | | |
|---|---|---|---|
| 0 | 1.3 | 2.1 | 96.6 |
| 15 | 2.2 | 2.5 | 95.2 |
| 30 | 3.4 | 3 | 93.6 |
| 60 | 4.9 | 3.6 | 91.5 |
| 120 | 8.2 | 3.5 | 88.3 |

40 mM, pH 3.2, 20 g/L, 30 C.

| | | | |
|---|---|---|---|
| 0 | 1.9 | 2.2 | 95.9 |
| 15 | 3.2 | 2.5 | 94.3 |
| 30 | 4.9 | 2.9 | 92.2 |
| 60 | 6.2 | 3.9 | 89.9 |
| 120 | 8.8 | 5.4 | 85.8 |

80 mM, pH 3.2, 40 g/L, 30 C.

| | | | |
|---|---|---|---|
| 0 | 20.9 | 5.6 | 73.5 |
| 15 | 33.9 | 8.4 | 57.4 |
| 30 | 40.3 | 9.9 | 49.5 |
| 60 | 41.3 | 11.1 | 47.5 |
| 120 | 43.9 | 13.9 | 41.3 |

80 mM, pH 3.2, 20 g/L, 30 C.

| | | | |
|---|---|---|---|
| 0 | 18.8 | 6.4 | 74.8 |
| 15 | 29.6 | 8.6 | 61.6 |
| 30 | 36.9 | 12.8 | 50.2 |
| 60 | 39.8 | 14.7 | 44.9 |
| 120 | 39.1 | 21.8 | 38.6 |

40 mM, pH 3.8, 20 g/L, 30 C.

| | | | |
|---|---|---|---|
| 0 | 0.4 | 0.9 | 98.7 |
| 15 | 0.4 | 1 | 98.6 |
| 30 | 0.5 | 1 | 98.5 |
| 60 | 0.6 | 1 | 98.4 |
| 120 | 0.4 | 0.9 | 98.7 |

40 mM, pH 3.8, 40 g/L, 30 C.

| | | | |
|---|---|---|---|
| 0 | 0.3 | 1 | 98.7 |
| 15 | 0.4 | 1 | 98.6 |
| 30 | 0.4 | 1 | 98.6 |
| 60 | 0.5 | 1.1 | 98.4 |
| 120 | 0.7 | 1.1 | 98.2 |

80 mM, pH 3.8, 40 g/L, 30 C.

| | | | |
|---|---|---|---|
| 0 | 1.2 | 1.6 | 97.2 |
| 15 | 2.4 | 1.9 | 95.7 |
| 30 | 2.6 | 1.7 | 95.7 |
| 60 | 5 | 2.2 | 92.8 |
| 120 | 7 | 2.3 | 90.7 |

80 mM, pH 3.8, 20 g/L, 30 C.

| | | | |
|---|---|---|---|
| 0 | 1.5 | 1.9 | 96.6 |
| 15 | 2.7 | 2.3 | 95 |
| 30 | 3 | 2.2 | 94.9 |
| 60 | 4.8 | 2.8 | 92.4 |
| 90 | | | |

### REFERENCES

(1) Shakula et al., “Strategies to address aggregation during Protein A chromatography,” Bioprocess International, pp. 36-44, (May 2005)
(2) Shakula et al., “Protein aggregation kinetics during Protein A chromatography case study for an Fc fusion protein,” Journal of Chromatography, 1171, pp. 22-28, (2007)
(3) Cromwell et al., “Protein Aggregation and Bioprocessing,” The AAPS Journal, 8(3), pp. E572-E579, (2006)
(4) Mezzasalma et al., “Enhancing recombinant protein quality and yield by protein stability profiling,” Journal of Biomolecular Screening, 12, pp. 418-428, (2007)
(5) Arakawa et al., “Elution of antibodies from a Protein A column by aqueous arginine solutions,” Protein Expression & Purification, 36, pp. 244-248, (2004)
(6) Arakawa et al., “Aggregation Analysis of Therapeutic Proteins, Part 1,” Bioprocess International, 4(10):42-43, (November 2006)
(7) FDA USDHHS, “Points to consider in the Manufacture and Testing of Monoclonal Antibody Products for Human Use,” Center for Biologics Evaluation and Research, (Feb. 28, 1997).
(8) FDA USDHHS, “Guidance for Industry: Q5A Viral Safety Evaluation of Biotechnology Products Derived From Cell Lines of Human or Animal Origin,” Center for Drug Evaluation of Research, ICH, Center for Biologics Evaluation and Research., (September 1998)
(9) Brorson, et al., “Bracketed generic inactivation of rodent retroviruses by low pH treatment for monoclonal antibodies and recombinant proteins,” Biotechnology and Bioengineering, v. 82, n.3, pp. 321-329, (May 5, 2003)
(10) See generally, Fundamental Immunology, Paul, W., ed., 2nd ed., Raven Press, N.Y., Ch. 7 (1989)
(11) U.S. Pat. No. 4,816,567
(12) U.S. patent application US 2005/0038231
(13) Tugcu et al., Biotechnology and Bioengineering, v. 99, pp. 599-613 (2007)
(14) PCT Intl. Application No. PCT/US2005/038125
(15) U.S. Ser. No. 11/581,931
(16) U.S. Ser. No. 12/012,885
(17) PCT Intl. Application No. PCT/US2006/040508
(18) U.S. Ser. No. 11/875,017
(19) Johnson, C. M., “Differential Scanning Calorimetry: Theory and Practice,” Micro Cal Application Notes, pp. 1-9, (2005)
(20) Acharaya, P., “Using DSC to make downstream purification processing more economically viable,” Micro Cal Application Notes, pp. 1-4, (2006)
(21) Acharaya, P., Structural studies as a screening tool in development of protein purification processes, Current trends in Microcalorimetry, Jul. 18-21, 2007 (Boston, Mass.).
(22) Wen et al., “Application of DSC for antibodies and Fc-conjugated proteins,” American Pharmaceutical Review, 10(6), pp 10-15, (2007); Wen, J, Studying antibodies and Fc related proteins by DSC, Current trends in Microcalorimetry, Jul. 18-21, 2007 (Boston, Mass.).
(23) Demarst, S., “Better guidance in antibody therapeutics process development using DSC,” Micro Cal Application Notes, pp. 1-3, (2005)
(24) Ionescu, et al., “Contribution of variable domains to the stability of humanized IgG1 monoclonal antibodies,” Journal of Pharmaceutical Sciences, Wiley Interscience, www.interscience.wiley.com [doi: 10.1002/jps.21104]
(25) Van Buren et al., “Elucidation of Two Major Aggregation Pathways in an IgG2 antibody'” Journal of Pharmaceutical Sciences, Wiley Interscience, www.interscience.wiley.com [doi: 10.1002/jps.21514]

What is claimed is:

1. A method for purifying a monomeric monoclonal antibody from a sample, wherein the sample comprises the monomeric monoclonal antibody, host cell impurities, dimers, and higher order aggregates, comprising:
a) contacting the sample with a Protein A affinity chromatography column;
b) eluting the monomeric monoclonal antibody from the Protein A affinity chromatography column with an elution buffer; and
c) collecting one or more fractions of the monomeric monoclonal antibody from step (b) to form a Protein A product pool, wherein the product pool
i) comprises less than 5% higher order aggregate, and
ii) has a pH from about 3.5 to about 4.5,
thereby purifying the monomeric monoclonal antibody from the sample.

2. The method according to claim 1, wherein the elution buffer is citrate or acetate.

3. The method according to claim 2, wherein the elution buffer is citrate.

4. The method according to claim 3, wherein the concentration of citrate in the elution buffer is from about 0.030 M to about 0.085 M.

5. The method according to claim 2, wherein the elution buffer is acetate.

6. The method according to claim 5, wherein the concentration of acetate in the elution buffer is from about 0.050 M to about 0.200 M.

7. The method according to claim 1, wherein the method is conducted at a temperature from about 4° C. to about 30° C.

8. The method according to claim 7, wherein the method is conducted at a temperature from about 15° C. to about 27° C.

9. The method according to claim 1, wherein the monomeric monoclonal antibody is an IgG antibody.

10. The method according to claim 9, wherein the monomeric monoclonal antibody is an IgG1 or a modified IgG2 antibody.

11. The method according to claim 10, wherein the monomeric monoclonal antibody is an IgG2m4 antibody.

12. The method according to claim 11, wherein the IgG2m4 antibody is an anti-DKK1 antibody.

13. The method according to claim 1, wherein an amino acid is added to the elution buffer in step (b) to a concentration of from about 50 mM to about 500 mM.

14. The method according to claim 13, wherein the amino acid is arginine, proline, or histidine.

15. A method for purifying a monomeric monoclonal antibody from a sample, wherein the sample comprises the monomeric monoclonal antibody, host cell impurities, dimers, and higher order aggregates, comprising:
a) contacting the sample with a Protein A affinity chromatographic column at a temperature from about 15° C. to about 27° C.;
b) eluting the monomeric monoclonal antibody from the Protein A affinity chromatographic column with an elution buffer comprising citrate at a concentration from about 0.030 M to about 0.085 M; and
c) collecting one or more fractions of the monomeric monoclonal antibody from step (b) to form a Protein A product pool, wherein the product pool
i) comprises less than 5% higher order aggregate, and
ii) has a pH from about 3.5 to about 4.0,
thereby purifying the monomeric monoclonal antibody from the sample.

16. A method for purifying a monomeric monoclonal antibody from a sample, wherein the sample comprises the monomeric monoclonal antibody, host cell impurities, dimers, and higher order aggregates, comprising:
a) contacting the sample with a Protein A affinity chromatographic column at a temperature from about 15° C. to about 27° C.;
b) eluting the monomeric monoclonal antibody from the Protein A affinity chromatographic column with an elution buffer comprising acetate at a concentration from about 0.050 M to about 0.200 M; and
c) collecting one or more fractions of the monomeric monoclonal antibody from step (b) to form a Protein A product pool, wherein the product pool
i) comprises less than 5% higher order aggregate, and
ii) has a pH from about 3.5 to about 4.5,
thereby purifying the monomeric monoclonal antibody from the sample.

17. A method for purifying a monomeric monoclonal antibody from a sample, wherein the sample comprises the monomeric monoclonal antibody, host cell impurities, dimers, and higher order aggregates, comprising:
a) contacting the sample with a Protein A affinity chromatography column;
b) eluting the monomeric monoclonal antibody from the Protein A affinity chromatography column with an elution buffer; and
c) collecting one or more fractions of the monomeric monoclonal antibody from step (b) to form a Protein A product pool, wherein the product pool
i) comprises less than 5% higher order aggregate, and
ii) has a pH from about 3.2 to about 4.5,
thereby purifying the monomeric monoclonal antibody from the sample.

18. A method for purifying a monomeric monoclonal antibody from a sample, wherein the sample comprises the monomeric monoclonal antibody, host cell impurities, dimers, and higher order aggregates, comprising:
a) contacting the sample with a Protein A affinity chromatographic column at a temperature from about 15° C. to about 27° C.;
b) eluting the monomeric monoclonal antibody from the Protein A affinity chromatographic column with an elution buffer comprising citrate at a concentration from about 0.030 M to about 0.085 M; and
c) collecting one or more fractions of the monomeric monoclonal antibody from step (b) to form a Protein A product pool, wherein the product pool
i) comprises less than 5% higher order aggregate, and
ii) has a pH from about 3.2 to about 4.0,
thereby purifying the monomeric monoclonal antibody from the sample.

19. A method for purifying a monomeric monoclonal antibody from a sample, wherein the sample comprises the monomeric monoclonal antibody, host cell impurities, dimers, and higher order aggregates, comprising:
a) contacting the sample with a Protein A affinity chromatographic column at a temperature from about 15° C. to about 27° C.;
b) eluting the monomeric monoclonal antibody from the Protein A affinity chromatographic column with an elution buffer comprising acetate at a concentration from about 0.050 M to about 0.200 M; and
c) collecting one or more fractions of the monomeric monoclonal antibody from step (b) to form a Protein A product pool, wherein the product pool
i) comprises less than 5% higher order aggregate, and
ii) has a pH from about 3.2 to about 4.5,
thereby purifying the monomeric monoclonal antibody from the sample.
